SEQUENCE LISTING (42 sequences)

SEQ ID NO: 1 - protein, 288 aa, Neisseria gonorrhoeae
MKTFFKTLSAAALALILAACGGQKDSAPAASAAAPSADNGAAKKEIVFGTTVGDFGDMVKEQIQAELEKKGYTVKLVEFTDYVRPNLALAEGELDINVFQHKPYLDDFKKEHNLDITEAFQVPTAPLGLYPGKLKSLEEVKDGSTVSAPNDPSNFARALVMLNELGWIKLKDGINPLTASKADIAENLKNIKIVELEAAQLPRSRADVDFAVVNGNYAISSGMKLTEALFQEPSFAYVNWSAVKTADKDSQWLKDVTEAYNSDAFKAYAHKRFEGYKYPAAWNEGAAK

SEQ ID NO: 2 - protein, 4 aa, Neisseria gonorrhoeae
GAME

SEQ ID NO: 3 - protein, 5 aa, Neisseria gonorrhoeae
KLAAA

SEQ ID NO: 4 - protein, 274 aa, Neisseria gonorrhoeae
GAMEKDSAPAASAAAPSADNGAAKKEIVFGTTVGDFGDMVKEQIQAELEKKGYTVKLVEFTDYVRPNLALAEGELDINVFQHKPYLDDFKKEHNLDITEAFQVPTAPLGLYPGKLKSLEEVKDGSTVSAPNDPSNFARALVMLNELGWIKLKDGINPLTASKADIAENLKNIKIVELEAAQLPRSRADVDFAVVNGNYAISSGMKLTEALFQEPSFAYVNWSAVKTADKDSQWLKDVTEAYNSDAFKAYAHKRFEGYKYPAAWNEGAAKKLAAA

SEQ ID NO: 5 - protein, 23 aa, Artificial Sequence
  recombinant signal sequence
HHHHHHDYDIPTTENLYFQGAME

SEQ ID NO: 6 - protein, 23 aa, Neisseria gonorrhoeae
MKTFFKTLSAAALALILAACGGQ

SEQ ID NO: 7 - DNA, 10 bp, Artificial Sequence
  consensus oligodeoxynucleotide
  misc_feature (1)..(3): n is any nucleotide
  misc_feature (4)..(4): n is T, G or A
  misc_feature (7)..(7): n is A or T
  misc_feature (8)..(10): n is any nucleotide
nnnncgnnnn

SEQ ID NO: 8 - DNA, 20 bp, Artificial Sequence
  immunostimulatory oligodeoxynucleotide
ataatcgacgttcaagcaag

SEQ ID NO: 9 - DNA, 13 bp, Artificial Sequence
  immunostimulatory oligodeoxynucleotide
ctcgagcgttctc

SEQ ID NO: 10 - DNA, 14 bp, Artificial Sequence
  immunostimulatory oligodeoxynucleotide
tctcgagcgttctc

SEQ ID NO: 11 - DNA, 16 bp, Artificial Sequence
  immunostimulatory oligodeoxynucleotide
actctggagcgttctc

SEQ ID NO: 12 - DNA, 12 bp, Artificial Sequence
  immunostimulatory oligodeoxynucleotide
tgcagcgttctc

SEQ ID NO: 13 - DNA, 12 bp, Artificial Sequence
  immunostimulatory oligodeoxynucleotide
tcgaggcttctc

SEQ ID NO: 14 - DNA, 12 bp, Artificial Sequence
  immunostimulatory oligodeoxynucleotide
gtcggcgttgac

SEQ ID NO: 15 - DNA, 19 bp, Artificial Sequence
  Immunostimultaroy oligodeoxynucleotide
tcgactctcgagcgttctc

SEQ ID NO: 16 - DNA, 20 bp, Artificial Sequence
  immunostimulatory oligodeoxynucleotide
atcgactctcgagcgttctc

SEQ ID NO: 17 - DNA, 12 bp, Artificial Sequence
  immunostimulatory oligodeoxynucleotide
tcgagcgttctc

SEQ ID NO: 18 - DNA, 12 bp, Artificial Sequence
  immunostimulatory oligodeoxynucleotide
gtcggcgtcgac

SEQ ID NO: 19 - DNA, 12 bp, Artificial Sequence
  immunostimulatory oligodeoxynucleotide
gtcgacgttgac

SEQ ID NO: 20 - DNA, 16 bp, Artificial Sequence
  immunostimulatory oligodeoxynucleotide
actctcgagggttctc

SEQ ID NO: 21 - DNA, 16 bp, Artificial Sequence
  immunostimulatory oligodeoxynucleotide
actctcgagcgttctc

SEQ ID NO: 22 - DNA, 12 bp, Artificial Sequence
  immunostimulatory oligodeoxynucleotide
gtcgtcgatgac

SEQ ID NO: 23 - DNA, 12 bp, Artificial Sequence
  immunostimulatory oligodeoxynucleotide
gtcgacgctgac

SEQ ID NO: 24 - DNA, 12 bp, Artificial Sequence
  immunostimulatory oligodeoxynucleotide
gtcgacgtcgac

SEQ ID NO: 25 - DNA, 12 bp, Artificial Sequence
  immunostimulatory oligodeoxynucleotide
gtcatcgatgca

SEQ ID NO: 26 - DNA, 12 bp, Artificial Sequence
  immunostimulatory oligodeoxynucleotide
gtcagcgtcgac

SEQ ID NO: 27 - DNA, 11 bp, Artificial Sequence
  immunostimulatory oligodeoxynucleotide
tcgagcgttct

SEQ ID NO: 28 - DNA, 16 bp, Artificial Sequence
  immunostimulatory oligodeoxynucleotide
actctggagcgttctc

SEQ ID NO: 29 - DNA, 16 bp, Artificial Sequence
  immunostimulatory oligodeoxynucleotide
actctcgagggttctc

SEQ ID NO: 30 - DNA, 16 bp, Artificial Sequence
  immunostimulatory oligodeoxynucleotide
actctcgagcgttcta

SEQ ID NO: 31 - DNA, 16 bp, Artificial Sequence
  immunostimulatory oligodeoxynucleotide
catctcgagcgttctc

SEQ ID NO: 32 - DNA, 14 bp, Artificial Sequence
  immunostimulatory oligodeoxynucleotide
actctttcgttctc

SEQ ID NO: 33 - DNA, 11 bp, Artificial Sequence
  immunostimulatory oligodeoxynucleotide
tcgagcgttct

SEQ ID NO: 34 - DNA, 12 bp, Artificial Sequence
  immunostimulatory oligodeoxynucleotide
tcgttcgttctc

SEQ ID NO: 35 - DNA, 15 bp, Artificial Sequence
  immunostimulatory oligodeoxynucleotide
gctagacgttagcgt

SEQ ID NO: 36 - DNA, 12 bp, Artificial Sequence
  immunostimulatory oligodeoxynucleotide
tcgaggcttctc

SEQ ID NO: 37 - DNA, 24 bp, Artificial Sequence
  immunostimulatory oligodeoxynucleotide
tcgtcgttttacggcgccgtgccg

SEQ ID NO: 38 - DNA, 24 bp, Artificial Sequence
  immunostimulatory oligodeoxynucleotide
tcgtcgttttgtcgttttgtcgtt

SEQ ID NO: 39 - DNA, 20 bp, Artificial Sequence
  immunostimulatory oligodeoxynucleotide
tccatgacgttcctgacgtt

SEQ ID NO: 40 - DNA, 20 bp, Artificial Sequence
  immunostimulatory oligodeoxynucleotide
gggggacgatcgtcgggggg

SEQ ID NO: 41 - DNA, 21 bp, Artificial Sequence
  immunostimulatory oligodeoxynucleotide
ggggacgacgtcgtggggggg

SEQ ID NO: 42 - DNA, 22 bp, Artificial Sequence
  Immunostimulatory oligodeoxynucleotide
tgactgtgaacgttcgagatga

(19) United States
(12) Patent Application Publication
(10) Pub. No.: US 2023/0241196 A1
(43) Pub. Date: Aug. 3, 2023

(54) GONORRHEA SUBUNIT VACCINE

(71) Applicants: Oregon State University, Corvallis, OR (US); The Henry M. Jackson Foundation for the Advancement of Military Medicine, Inc, Rockville, MD (US)

(72) Inventors: Aleksandra E. Sikora, Albany, OR (US); Ryszard A. Zielke, Corvallis, OR (US); Ann E. Jerse, Bethesda, MD (US)

(73) Assignees: Oregon State University, Corvallis, OR (US); The Henry M. Jackson Foundation for the Advancement of Military Medicine, Inc., Rockville, MD (US)

(21) Appl. No.: 17/790,360

(22) PCT Filed: Jan. 26, 2021
(86) PCT No.: PCT/US2021/015117

§ 371 Date: Jun. 30, 2022

Related U.S. Application Data
(60) Provisional application No. 62/966,179, filed on Jan. 27, 2020.

(51) Int. Cl. A61K 39/095; A61P 31/04

(52) U.S. Cl. CPC A61K 39/095; A61P 31/04; A61K 2039/55561

(57) ABSTRACT

Methods are disclosed for inducing an immune response to Neisseria gonorrhoeae in a mammalian subject. These methods include administering to the mammalian subject an effective amount of a MetQ protein and an effective amount of a K-type CpG oligodeoxynucleotide, thereby inducing the immune response. Also disclosed are immunogenic compositions including an effective amount of a MetQ protein and an effective amount of a K-type CpG oligodeoxynucleotide.

### CROSS REFERENCE TO RELATED APPLICATIONS

This claims the benefit of U.S. Provisional Application No. 62/966,179, filed Jan. 27, 2020, which is incorporated herein by reference.

### STATEMENT OF GOVERNMENT SUPPORT

This invention was made with Government support under grant number R01-AI117235 by the National Institutes of Health, the National Institute of Allergy and Infectious Diseases. The United States Government has certain rights in the invention.

### FIELD OF THE DISCLOSURE

This relates to immunogenic compositions comprising a MetQ protein and an CpG oligodeoxynucleotide (ODN), that are of use to induce an immune response to Neisseriagonorrhoeae.

### BACKGROUND

Vaccines against infectious diseases have indisputably transformed human health. In light of omnipresent antibiotic resistance, there is a huge need for continuous efforts to develop vaccines against challenging bacterial infections, including the highly prevalent and drug-resistant disease, gonorrhea. The causative organism, Neisseriagonorrhoeae (Ng) is a Gram-negative diplococcus that exclusively plagues humankind, yielding about 87 million new cases annually across the globe (Rowley et al., Bull World Health Organ 97, 548-562P (2019)). In the United States, it remains the second most commonly reported notifiable disease and the number of cases has risen steadily since the historic low in 2009, increasing by 82.6% (a total of 583,405 reported cases) in 2018. Gonorrhea is a sexually transmitted infection that can also be vertically transmitted to newborns during delivery (Magidson et al., J Psychosom Res 76, 322-328 (2014)). The consequences of the disease have devastating health and psychological impacts that profoundly affect quality of life (Magidson et al., J Psychosom Res 76, 322-328 (2014)). Clinical presentations vary between infection site and gender and include cervicitis, urethritis, proctitis, conjunctivitis, or pharyngitis. Women tend to encounter more frequent asymptomatic gonococcal infections and serious long-term reproductive health problems including endometritis, pelvic inflammatory disease, pregnancy complications and infertility (Rice et al., Annual review of microbiology 71, 665-686 (2017)). Neonates can be infected during delivery and most typically develop ophthalmia neonatorum but localized infections of other mucosal surfaces can also occur (Lochner and Maraqa, Pediatr Drugs 20, 501-509 (2018)). Additional complications associated with gonorrhea in patients may include musculoskeletal manifestations, such as suppurative arthritis, or the distinct syndrome associated with disseminated infection, which includes tenosynovitis, skin lesions, and polyarthralgia (Rice, Infect Dis Clin North Am 19, 853-861 (2005)). The grave nature of gonorrhea infections is further exacerbated by its ability to augment HIV infectivity and patient susceptibility to HIV (Fleming and Wasserheit, Sex Transm Infect 75, 3-17 (1999)). In the United States, patients presenting with uncomplicated Neisseriagonorrhoeae infections are given a single dose of injectable ceftriaxone and oral azithromycin in compliance with treatment guidelines from the Centers for Disease Control and Prevention (CDC, 2015). Alarmingly, however, these antimicrobials are rapidly losing their effectiveness (Fifer et al., N Engl J Med 374, 2504-2506 (2016); Eyre et al., Euro surveillance : bulletin Europeen sur les maladies transmissibles = European communicable disease bulletin 23, (2018); England, Health Protection Report (2018)). A need remains for vaccines that can be used to induce an immune response to gonorrhea infection, such as a protective immune response.

### SUMMARY OF THE DISCLOSURE

Methods are disclosed for inducing an immune response to Neisseriagonorrhoeae in a mammalian subject. These methods include administering to the mammalian subject an effective amount of a MetQ protein and an effective amount of a K-type CpG oligodeoxynucleotide, thereby inducing the immune response.

Also disclosed are immunogenic compositions including an effective amount of a MetQ protein and an effective amount of a K-type CpG oligodeoxynucleotide.

The foregoing and other features and advantages of the invention will become more apparent from the following detailed description of several embodiments which proceeds with reference to the accompanying figures.

### BRIEF DESCRIPTION OF THE FIGURES

FIGS. 1A-1C. Conservation of Neisseriagonorrhoeae (Ng) MetQ and mapped amino acid polymorphic sites (A) A maximum likelihood phylogenetic tree was constructed for metQ alleles in MEGA using the Jones-Taylor-Thornton model to generate a pairwise distance matrix, to which Neighbor-Join and BioNJ algorithms were applied to obtain the initial tree for a heuristic search. Phylogenies were tested with 500 bootstrap iterations, and the tree with the highest log-likelihood is presented. The FA1090 allele is highlighted. (B, C) Ng MetQ amino acid polymorphisms were mapped to crystal structures of Nm MetQ using PyMol. Structures from proteins isolated under standard conditions (PDB ID: 3IR1; B) or under D-methionine conditions (PDB ID: 3XGA; C) are presented. Sulfate ions are associated with both structures but are presented only in panel (C) for clarity. L-methionine is in the MetQ active site in both structures.

FIGS. 2A-2D. MetQ is expressed during Ng infection in the murine lower reproductive tract but is dispensable for bacterial fitness. (A) Rapidly growing liquid cultures of strains indicated above the graph were standardized, diluted, and spotted onto solid media for standard growth conditions (SGC), iron limiting conditions (- Fe), presence of normal human serum (NHS), anaerobic conditions (- O₂), and anaerobic conditions combined with iron limitation (- O₂ - Fe). CFUs were scored following 22 h incubation. n = 3; mean± SEM. (B) Vaginal washes from five female experimentally infected BALB/c mice inoculated with WT Ng FA1090 were collected on days 1, 3, and 5 post-inoculation in two independent experiments and pooled washes from each experiment were probed with anti-MetQ antiserum. The amount of sample loaded onto SDS-PAGE was normalized based on the number of gonococcal CFUs recovered from the washes. The relative intensities of MetQ abundance were compared to the amount of MetQ on day 1 post-infection, which was arbitrarily set to 1. (C, D) Competitive infections between WT bacteria and either the ΔmetQ mutant (C) or the complementation strain ΔmetQ/P_{lac}::metQ (D) were performed by inoculating female BALB/c mice intravaginally with approximately equal numbers of each strain (~10⁶ CFU/dose). Vaginal swabs were collected on days 1, 3, and 5 post-infection and enumerated on medium containing streptomycin (total bacteria) or streptomycin and kanamycin (mutant bacteria). The competitive index (CI) was calculated as described in the main text. Experiments were performed on two separate occasions with six mice per group, and the geometric mean of the CI is presented. The assay’s limit of detection of 1 CFU was assigned for any strain not recovered from an infected mouse. Statistical analysis was executed using Kruskal-Wallis and Dunn’s multiple comparison tests to compare statistical significance of CIs between ΔmetQ/WT and ΔmetQ/P_{lac}: : metQ/WT competitions. Symbols designate mice from which no WT bacteria were recovered, while open symbols signify that no mutant CFU were recovered.

FIGS. 3A-3C. Experimental design of immunization/challenge experiments and MetQ antigen design. (A) To generate rMetQ antigen, the metQ gene, encoding a full-length MetQ protein, was engineered to produce a recombinant protein that lacks the signal sequence and carries an N-terminal 6xHistag followed by a Tobacco Etch Virus (TEV) protease cleavage site. (B) Soluble rMetQ was purified to homogeneity through several chromatography steps and migrated on SDS-PAGE at approximately 29.87 kDa, consistent with the predicted molecular mass of mature MetQ, as revealed by SYPRO Ruby staining. Untagged rMetQ was used in immunization studies as shown in panel C. (C) Female BALB/c mice were randomized into three experimental groups (n = 20/group) and given rMetQ-CpG, CpG, or PBS (unimmunized) subcutaneously on day 0, followed by three nasal boosts on days 14, 24 and 35. Vaginal washes were collected 10 days after the second immunization (d34) and serum was collected after the final immunization (day 49) to avoid disruption of the vaginal microenvironment prior to bacterial challenge. Three weeks after the final immunization (d 56 or d-2), only mice that entered into the diestrus or anestrus stage (n - number of animals indicated) were treated with 17β-estradiol and antibiotics and challenged with 10⁶ CFU of Ng FA1090 two days later (day 0). Vaginal swabs were quantitatively cultured for Ng on days 1, 3, 5, and 7 post-bacterial inoculation.

FIGS. 4A-4D. MetQ-specific serum IgG and vaginal IgG and IgA are induced by rMetQ-CpG. Female mice were immunized with rMetQ-CpG, CpG, or PBS in immunization/challenge studies. Total cell envelope (CE) proteins from Ng FA1090 and rMetQ were fractionated by SDS-PAGE. Immunoblotting was performed with pooled serum (A, B) and vaginal washes (C, D) collected after the third immunization, followed by secondary antibodies against mouse IgG (A, C) or IgA (B, D). The intensity of the bands and the value is recorded under each lane. ND-not detected.

FIG. 5. Anti-MetQ antibody responses elicited by rMetQ-CpG immunization. Post-immunization (d49) total IgG, IgGl, IgG2a, and IgA antibody titers in mice immunized with rMetQ-CpG, CpG, or unimmunized. Bar graphs represent geometric mean ELISA titers with error bars showing 95% confidence limits. Numbers above the bar graphs indicate reciprocal geometric mean ELISA titer values ×10³. Statistical significance between data in groups was determined using Kruskal-Wallis with Dunn’s multiple comparison test. ****p<0.0001; ***p=0.0007, **p=0.002, *p=0.024.

FIGS. 6A-6C. Mice immunized with rMetQ-CpG clear infections significantly faster following vaginal challenge with Ng. Groups of BALB/c mice were immunized with rMetQ-CpG or given CpG (adjuvant) alone or PBS (unimmunized) as per the immunization regimen shown in FIG. 3. Subsequently, mice in the diestrus stage or in anestrus were treated with 17β-estradiol and antibiotics and challenged with 10⁶ CFU of strain FA1090 three weeks after the final immunization (n=16-18 mice/group). Vaginal washes were quantitatively cultured for N.gonorrhoeae on days 1, 3, 5 and 7 post-bacterial challenge. (A) The percentage of mice with positive vaginal cultures was plotted over time as Kaplan Meier curves and the results analyzed by the Log Rank test. (B) The average number of CFU recovered from each experimental group was plotted over time. The limit of detection was 20 CFU/mL of vaginal swab suspension. This value was used for mice with negative cultures. (C) AUC (log₁₀ CFU/mL) analysis of murine colonization. Data are presented for individual mice. Horizontal bars represent the geometric mean of the data with the 95% confidence interval. Data shown are combined data from two independent experiments.

FIGS. 7A-7F. Infection dynamics from individual rMetQ-CpG immunization experiments. Groups of BALB/c mice were immunized with rMetQ-CpG or given CpG (adjuvant) alone or PBS (unimmunized) as per the immunization regimen shown in FIG. 3. Subsequently, mice in the diestrus stage or in anestrus were treated with 17β-estradiol and antibiotics and challenged with 10⁶ CFU of strain FA1090 three weeks after the final immunization (n=16-18 mice/group). Vaginal washes were quantitatively cultured for N.gonorrhoeae on days 1, 3, 5 and 7 post-bacterial challenge. Individual experiments are presented in panels (A, B, C) and (D, E, F), and correspond to the combined data presented in FIG. 6. (A, D) The percentage of mice with positive vaginal cultures was plotted over time as Kaplan Meier curves and the results analyzed by the Log Rank test. (B, E) The average number of CFU recovered from each experimental group was plotted over time. The limit of detection was 20 CFU/mL of vaginal swab suspension. This value was used for mice with negative cultures. (C, F) AUC (log₁₀ CFU/mL) analysis of murine colonization. Data are presented for individual mice. Horizontal bars represent the geometric mean of the data with the 95% confidence interval.

FIG. 8 is an alignment showing a recombinant MetQ (SEQ ID NO: 4) genetically modified to be optimally produced in a heterologous host (E.coli). Briefly, the natural signal peptide (SEQ ID NO: 6, removed during protein maturation) was removed, histidine tag was added and the signal peptide was replaced with a sequence (SEQ ID NO: 5) that is recognized by TEV protease. This allowed the histidine tag to be removed, to optimize the immune response to specifically target MetQ. Further, the natural MetQ has a sequence CGGQ (20-23 of SEQ ID NO: 1) with the first cysteine comprising site where the antigen is anchored to bacterial membrane. The sequence GAME (SEQ ID NO: 2) sequence was added, and KLAAA (SEQ ID NO: 3 was added at the 3′ end of protein. In this figure, the variable sequence (combination of X’s, J and Z) are either

HHHHHHDYDIPTTENLYFQGAME

(SEQ ID NO: 5, the recombinant sequence) or

MKTFFKTLSAAALALILAACGGQ

(SEQ ID NO: 6, the natural signal sequence). The immunogen of SEQ ID NO: 4 is shown. J is either leucine (L) or isoleucine (I) ; Z is either glutamic acid (E) or glutamine (Q). These amino acid pairs are very similar. J and Z were determined from the comparison of SEQ ID NO: 5 with SEQ ID NO: 6.

In the alignment, shown are, in N- to C-terminal order, a N-terminal methionine, e.g., in “MSS,” SEQ ID NO: 5 or SEQ ID NO: 6, SEQ ID NO: 1, and then SEQ ID NO: 3. When SEQ ID NO: 6 is present, it can be removed by the TEV protease.

FIGS. 9A-9B are an Alignment of the amino acid sequences of the allele 10 (N. g. FA1090 MetQ) aligned with the other alleles found in other Ng isolates. SEQ ID NO: 1 is shown as the consensus sequence.

FIG. 10 is a Table showing a Comparison of MetQ alleles distribution in 4,411 isolates of Neisseriagonorrhoeae. (AA - amino acid).

FIGS. 11A-11C. Infection dynamics from individual rMetQ-CpG immunization experiments. Groups were given rMetQ, rMetQ-CpG, CpG, or PBS (A, B, C). Mice in the diestrus stage or in anestrus were treated with 17β-estradiol and antibiotics and challenged with 10⁶ CFU of strain FA1090 three weeks after the final immunization. Vaginal washes were quantitatively cultured for N.gonorrhoeae on days 1, 3, 5 and 7 post-bacterial challenge. (A) The percentage of mice with positive vaginal cultures was plotted over time as Kaplan Meier curves and the results analyzed by the Log Rank test. (B) The average number of CFU recovered from each experimental group was plotted over time. The limit of detection was 20 CFU/mL of vaginal swab suspension. This value was used for mice with negative cultures. Differences in colonization load were assessed by a repeated-measures two-way analysis of variance (ANOVA) using Bonferroni’s post hoc analysis for multiple pairwise comparisons (C) AUC (log10 CFU/mL) analysis of murine colonization. Data are presented for individual mice. Horizontal bars represent the geometric mean of the data with the 95% confidence interval.

FIGS. 12A-12B. Mice immunized with NGO1985-CpG do not clear infections significantly faster following vaginal challenge with Ng. Groups of BALB/c mice were immunized with NGO1985-CpG or given CpG (adjuvant) alone or PBS (unimmunized). Subsequently, mice in the diestrus stage or in anestrus were treated with 17β-estradiol and antibiotics and challenged with 10⁶ CFU of strain FA1090 three weeks after the final immunization (n=16-18 mice/group). Vaginal washes were quantitatively cultured for N.gonorrhoeae on days 1, 3, 5 and 7 post-bacterial challenge. (A) The percentage of mice with positive vaginal cultures was plotted over time as Kaplan Meier curves and the results analyzed by the Log Rank test. (B) The average number of CFU recovered from each experimental group was plotted over time. The limit of detection was 20 CFU/mL of vaginal swab suspension. This value was used for mice with negative cultures. Differences in colonization load were assessed by a repeated-measures two-way analysis of variance (ANOVA) using Bonferroni’s post hoc analysis for multiple pairwise comparisons. There were no statistically significant differences between any experimental groups.

### SEQUENCE LISTING

The nucleic and amino acid sequences listed in the accompanying sequence listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. The Sequence Listing is submitted as an ASCII text file Sequence_Listing.txt, dated Jan. 25, 2021, sized 14KB, which is incorporated by reference herein. In the accompanying sequence listing:

SEQ ID NO: 1 is the amino acid sequence of a consensus Neisseria gonorrhoeae MetQ protein.

SEQ ID NO: 2 is a synthetic sequence of a MetQ immunogen.

SEQ ID NO: 3 is a synthetic sequence of a MetQ immunogen.

SEQ ID NO: 4 is the amino acid sequence of a recombinant MetQ immunogen.

SEQ ID NO: 5 is the amino acid sequence of a recombinant protein including a sequence recognized by TEV proteins.

SEQ ID NO: 6 the amino acid sequence of a MetQ signal sequence.

SEQ ID NOs: 7-42 are the nucleic acid sequences of K-type ODNs.

### DETAILED DESCRIPTION OF SEVERAL EMBODIMENTS

MetQ is a highly conserved, surface-displayed lipoprotein. It is disclosed herein that MetQ was used as a component of a gonorrhea subunit vaccine. Subunit antigens are appealing candidates for vaccine development due to their safety, reduced chance of side effects, cost-effectiveness, and rapid preparation (13, 14). MetQ was identified by proteomics- and genomics-based reverse vaccinology antigen discovery programs applied to Neisseriagonorrhoeae (12, 15, 16) and N.meningitidis (12, 15-17), respectively. High-throughput proteomic investigations and immunoblotting analyses demonstrated that MetQ is ubiquitously expressed in 36 geographically, temporally and genetically diverse Neisseriagonorrhoeae isolates, including in the 2016 panel of World Health Organization (WHO) Ng strains, in host-relevant growth conditions, and is present in naturally released outer membrane vesicles. MetQ, in addition to a traditional lipoprotein signal peptide, contains a NlpA domain homologous to the respective domain in the E.coli methionine binding protein MetQ (NlpA). Indeed, Neisseriagonorrhoeae MetQ binds L-methionine with nanomolar affinity. In addition to its function in methionine import, MetQ impacts Neisseriagonorrhoeae adhesion and invasion of epithelial cells and bacterial survival in primary monocytes, macrophages, and human serum (18). MetQ conservation was assessed on a large scale. It was determined that MetQ has protective efficacy in a female mouse model of lower genital tract infection when formulated with a T helper (Th) 1 response-inducing adjuvant (K-type CpG olidgodeoxynucleotide (ODN)). Thus, MetG can be used with a K-type CpG ODN to induce an immune response to Neisseriagonorrhoeae.

### Summary of Terms

Unless otherwise noted, technical terms are used according to conventional usage. Definitions of many common terms in molecular biology may be found in Krebs et al. (eds.), Lewin’s genes XII, published by Jones & Bartlett Learning, 2017. As used herein, the singular forms “a,” “an,” and “the,” refer to both the singular as well as plural, unless the context indicates otherwise. For example, the term “an antigen” includes single or plural antigens and can be considered equivalent to the phrase “at least one antigen.” As used herein, the term “comprises” means “includes.” Unless otherwise indicated “about” indicates within five percent. It is further to be understood that any and all base sizes or amino acid sizes, and all molecular weight or molecular mass values, given for nucleic acids or polypeptides are approximate, and are provided for descriptive purposes, unless otherwise indicated. Although many methods and materials similar or equivalent to those described herein can be used, particular suitable methods and materials are described below. In case of conflict, the present specification, including explanations of terms, will control. In addition, the materials, methods, and examples are illustrative only and not intended to be limiting. To facilitate review of the various embodiments, the following explanations of terms are provided:

Administration: The introduction of a composition into a subject by a chosen route. Administration can be local or systemic. For example, if the chosen route is intranasal, the composition is administered by introducing the composition into the nasal passages of the subject. Similarly, if the chosen route is intramuscular, the composition is administered by introducing the composition into a muscle of the subject. If the chosen route is oral, the composition is administered by introducing the subject ingesting the composition. Exemplary routes of administration of use in the methods disclosed herein include, but are not limited to, oral, injection (such as subcutaneous, intramuscular, intradermal, intraperitoneal, and intravenous), sublingual, rectal, transdermal (for example, topical), intranasal, vaginal, and inhalation routes.

Animal: Living multi-cellular vertebrate organisms, a category that includes, for example, mammals and birds. The term mammal includes both human and non-human mammals. Similarly, the term “subject” includes both human and veterinary subjects.

Amino acid substitution: The replacement of an amino acid in a polypeptide with one or more different amino acids. In the context of a protein sequence, an amino acid substitution is also referred to as a mutation.

Control: A reference standard. In some embodiments, the control is a negative control sample obtained from a healthy patient, or a subject immunized with a carrier. In other embodiments, the control is a positive control sample obtained from a patient immunized with a vaccine. In still other embodiments, the control is a historical control or standard reference value or range of values (such as a previously tested control sample, such as a group of patients with known prognosis or outcome, or group of samples that represent baseline or normal values).

A difference between a test sample and a control can be an increase or conversely a decrease. The difference can be a qualitative difference or a quantitative difference, for example a statistically significant difference. In some examples, a difference is an increase or decrease, relative to a control, of at least about 5%, such as at least about 10%, at least about 20%, at least about 30%, at least about 40%, at least about 50%, at least about 60%, at least about 70%, at least about 80%, at least about 90%, at least about 100%, at least about 150%, at least about 200%, at least about 250%, at least about 300%, at least about 350%, at least about 400%, at least about 500%, or greater than 500%.

Conservative variant: “Conservative” amino acid substitutions are those substitutions or deletions that do not substantially affect or decrease a function of a protein, such as the ability of the protein to elicit an immune response when administered to a subject. The term conservative amino acid substitution also includes the use of a substituted amino acid in place of an unsubstituted parent amino acid. Furthermore, individual substitutions, deletions or additions which alter, add or delete a single amino acid or a small percentage of amino acids (for instance less than 5%, in some embodiments less than 1%) in an encoded sequence are conservative variations where the alterations result in the substitution of an amino acid with a chemically similar amino acid.

The following six groups are examples of amino acids that are considered to be conservative substitutions for one another:
1) Alanine (A), Serine (S), Threonine (T);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (W).

Non-conservative substitutions are those that reduce an activity or function of the recombinant Env protein, such as the ability to elicit an immune response when administered to a subject. For instance, if an amino acid residue is essential for a function of the protein, even an otherwise conservative substitution may disrupt that activity. Thus, a conservative substitution does not alter the basic function of a protein of interest.

Consists essentially of and Consists Of: A polypeptide comprising an amino acid sequence that consists essentially of a specified amino acid sequence does not include any additional amino acid residues. However, the residues in the polypeptide can be modified to include non-peptide components, such as labels (for example, fluorescent, radioactive, or solid particle labels), sugars or lipids, and the N- or C-terminus of the polypeptide can be joined (for example, by peptide bond) to heterologous amino acids, such as a cysteine (or other) residue in the context of a linker for conjugation chemistry. A polypeptide that consists of a specified amino acid sequence does not include any additional amino acid residues, nor does it include additional biological components, such as nucleic acids lipids, sugars, nor does it include labels. However, the N- or C-terminus of the polypeptide can be joined (for example, by peptide bond) to heterologous amino acids, such as a peptide tag, or a cysteine (or other) residue in the context of a linker for conjugation chemistry.

A polypeptide that consists or consists essentially of a specified amino acid sequence can be glycosylated or have an amide modification. A polypeptide that consists of or consists essentially of a particular amino acid sequence can be linked via its N- or C-terminus to a heterologous polypeptide, such as in the case of a fusion protein containing a first polypeptide consisting or a first sequence that is linked (via peptide bond) to a heterologous polypeptide consisting of a second sequence. In another example, the N- or C-terminus of a polypeptide that consists of or consists essentially of a particular amino acid sequence can be linked to a peptide linker (via peptide bond) that is further linked to one or more additional heterologous polypeptides. In a further example, the N- or C-terminus of a polypeptide that consists of or consists essentially of a particular amino acid sequence can be linked to one or more amino acid residues that facilitate further modification or manipulation of the polypeptide.

CpG or CpG motif: A nucleic acid having a cytosine followed by a guanine linked by a phosphate bond in which the pyrimidine ring of the cytosine is unmethylated. The term “methylated CpG” refers to the methylation of the cytosine on the pyrimidine ring, usually occurring at the 5-position of the pyrimidine ring. A CpG motif is a pattern of bases that include an unmethylated central CpG surrounded by at least one base flanking (on the 3′ and the 5′ side of) the central CpG. Without being bound by theory, the bases flanking the CpG confer a significant part of the activity to the CpG oligodeoxynucleotide. A CpG oligodeoxynucleotide is an oligodeoxynucleotide that is at least about ten nucleotides in length and includes an unmethylated CpG. CpG oligodeoxynucleotides include both D -type (also known as A type) and K-type oligodeoxynucleotides (see below). CpG oligodeoxynucleotides are single-stranded. The entire CpG oligodeoxynucleotide can be unmethylated or portions may be unmethylated. In one embodiment, at least the C of the 5′ CG 3′ is unmethylated. K-type ODNs are linear, single-stranded polynucleotides that include unmethylated CpG motifs, and act through the Toll-like receptor (TLR)9 to induce NF-κB-dependent proinflammatory response characterized by the production of IL-6 and TNF-α by plasmacytoid dendritic cells (pDCs). K-type ODNs stimulate B cells to proliferate and secrete IgM. K-type CpG ODN nucleic acid sequences represented by the formula: 5′-N₁DCGYN₂-3′, wherein at least one nucleotide separates consecutive CpGs; D is adenine, guanine, or thymidine; Y is cytosine or thymine, N is any nucleotide and N₁ + N₂ is from about 0-26 bases.

Degenerate variant: In the context of the present disclosure, a “degenerate variant” refers to a polynucleotide encoding a polypeptide that includes a sequence that is degenerate as a result of the genetic code. There are 20 natural amino acids, most of which are specified by more than one codon. Therefore, all degenerate nucleotide sequences encoding a peptide are included as long as the amino acid sequence of the peptide encoded by the nucleotide sequence is unchanged.

Effective amount: An amount of agent, such as an immunogen, such as a composition including a MetQ protein and an adjuvant, that is sufficient to elicit a desired response, such as an immune response in a subject. In some embodiments, to obtain a protective immune response against an organism of interest, such as Neisseriagonorrhoeae, can require multiple administrations, and/or administration as the “prime” in a prime boost protocol. Accordingly, an effective amount of MetQ and a CpG ODN can be the amount sufficient to elicit a priming immune response in a subject that can be subsequently boosted to elicit a protective immune response.

In one example, a desired response is to inhibit or reduce or prevent a Neisseriagonorrhoeae infection. The Neisseriagonorrhoeae infection does not need to be completely eliminated or reduced or prevented for the method to be effective. For example, administration of an effective amount of the agent can decrease the Neisseriagonorrhoeae infection (for example, as measured by bacteria number or by number or percentage of subjects infected by Neisseriagonorrhoeae) by a desired amount, for example by at least 50%, at least 60%, at least 70%, at least 80%, at least 90%, at least 95%, at least 98%, or even at least 100% (elimination or prevention of detectable hPIV infection), as compared to a suitable control.

Epitope: An antigenic determinant. These are particular chemical groups or peptide sequences on a molecule that are antigenic, such that they elicit a specific immune response, for example, an epitope is the region of an antigen to which B and/or T cells respond. An antibody can bind to a particular antigenic epitope, such as an epitope presented on a microvesicle of Neisseriagonorrhoeae or Neisseriameningitidis. Epitopes can be formed both from contiguous amino acids or noncontiguous amino acids juxtaposed by tertiary folding of a protein.

Gonorrhea: A sexually transmitted infection (STI) caused by the bacterium Neisseriagonorrhoeae. Infection can involve the genitals, mouth, and/or rectum. Gonorrhea is spread through sexual contact with an infected person. Gonorrhea if left untreated may last for weeks or months with higher risks of complications. One of the complications of gonorrhea is systemic dissemination resulting in skin pustules or petechia, septic arthritis, meningitis, or endocarditis. In men, inflammation of the epididymis, prostate gland, and urethra can occur. In women, the most common result of untreated gonorrhea is pelvic inflammatory disease. Other complications include inflammation of the tissue surrounding the liver, septic arthritis in the fingers, wrists, toes, and ankles; septic abortion; chorioamnionitis during pregnancy; neonatal or adult blindness from conjunctivitis; and infertility.

Heterologous: Originating from a different genetic source, so that the biological components that are not found together in nature or that is synthetic. The components may be host cells, genes, or regulatory regions, such as promoters. Although the heterologous components are not found together in nature, they can function together, as when a promoter heterologous to a gene is operably linked to the gene.

Immune response: A response of a cell of the immune system, such as a B cell or T cell to a stimulus. In one embodiment, the response is specific for a particular antigen (an “antigen-specific response”). A “parameter of an immune response” is any particular measurable aspect of an immune response, including, but not limited to, cytokine secretion (IL-6, IL-10, IFNγ, etc.), immunoglobulin production, dendritic cell maturation, and proliferation of a cell of the immune system. One of skill in the art can readily determine an increase in any one of these parameters, using known laboratory assays. In one specific non-limiting example, to assess cell proliferation, incorporation of ³H-thymidine can be assessed. A “substantial” increase in a parameter of the immune response is a significant increase in this parameter as compared to a control. Specific, non-limiting examples of a substantial increase are at least about a 50% increase, at least about a 75% increase, at least about a 90% increase, at least about a 100% increase, at least about a 200% increase, at least about a 300% increase, and at least about a 500% increase.

A “protective immune response” is an immune response that confers protection against a disease caused by Neisseriagonorrhoeae. A “therapeutic immune response” treats an existing infection with Neisseriagonorrhoeae. In some embodiments, the subject has a Neisseriagonorrhoeae infection, and administration of the immunogenic composition increases clearance of Neisseriagonorrhoeae.

Immunogen: A compound, composition, or substance (for example, a composition including a MetQ protein and a CpG ODN) that can elicit an immune response in an animal, including compositions that are injected or absorbed into an animal. Administration of an immunogen to a subject can lead to immunity against a pathogen of interest, such as Neisseriagonorrhoeae.

Immunogenic composition: A composition comprising a MetQ protein and a CpG ODN that induces a measurable CTL response against Neisseriagonorrhoeae, or induces a measurable B cell response (such as production of antibodies) against Neisseriagonorrhoeae, when administered to a subject. For in vivo use, the immunogenic composition will typically include the MetQ protein and/or the CpG ODN in a pharmaceutically acceptable carrier and the adjuvant. The phrase “in an effective amount to elicit an immune response” means that there is a detectable difference between an immune response indicator measured before and after administration of a particular immunogenic composition. Immune response indicators include but are not limited to: antibody titer or specificity, as detected by an assay such as enzyme-linked immunosorbent assay (ELISA), bactericidal assay, flow cytometry, immunoprecipitation, Ouchterlony immunodiffusion; binding detection assays of, for example, spot, western blot or antigen arrays; cytotoxicity assays, etc.

Inhibiting or treating a disease: Inhibiting the full development of a disease or condition, for example, in a subject who is at risk for a disease such as a Neisseriagonorrhoeae infection. “Treatment” refers to a therapeutic intervention that ameliorates a sign or symptom of a disease or pathological condition after it has begun to develop. The term “ameliorating,” with reference to a disease or pathological condition, refers to any observable beneficial effect of the treatment. Inhibiting a disease can include preventing or reducing the risk of the disease, such as preventing or reducing the risk of bacterial infection. The beneficial effect can be evidenced, for example, by a delayed onset of clinical symptoms of the disease in a susceptible subject, a reduction in severity of some or all clinical symptoms of the disease, a slower progression of the disease, a reduction in the bacterial load, an improvement in the overall health or well-being of the subject, or by other parameters that are specific to the particular disease. A “prophylactic” treatment is a treatment administered to a subject who does not exhibit signs of a disease or exhibits only early signs for the purpose of decreasing the risk of developing pathology.

Isolated: An “isolated” biological component (such as a nucleic acid, peptide or protein) has been substantially separated, produced apart from, or purified away from other biological components in the cell of the organism in which the component naturally occurs, i.e., other chromosomal and extrachromosomal DNA and RNA, and proteins. Nucleic acids, peptides and proteins which have been “isolated” thus include nucleic acids and proteins purified by standard purification methods. The term also embraces nucleic acids, peptides and proteins prepared by recombinant expression in a host cell as well as chemically synthesized nucleic acids.

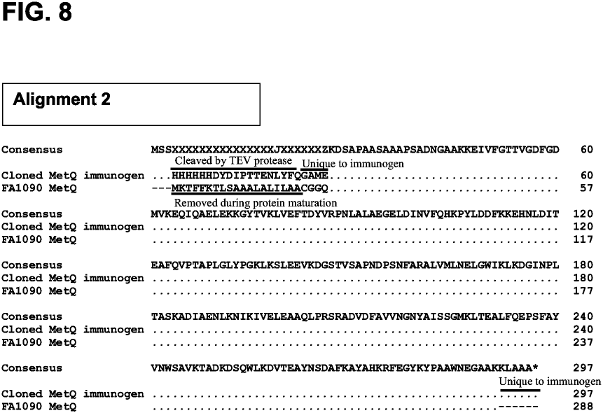

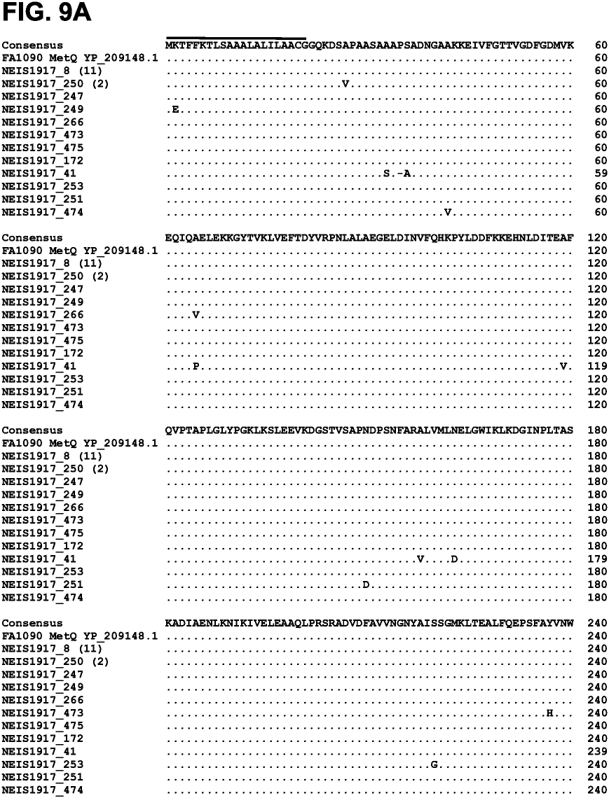

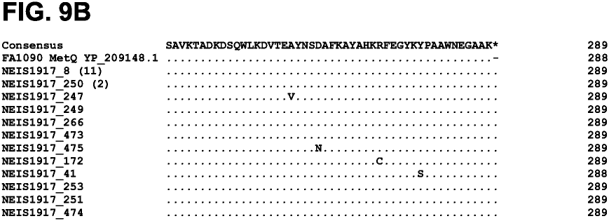

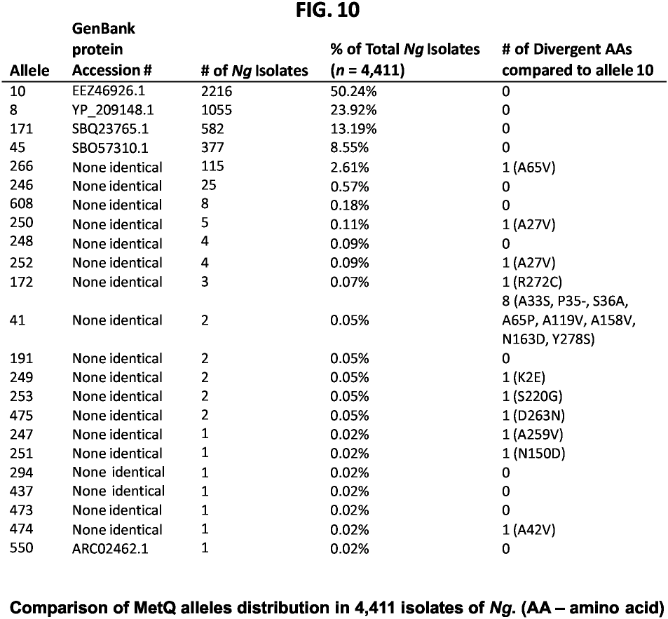

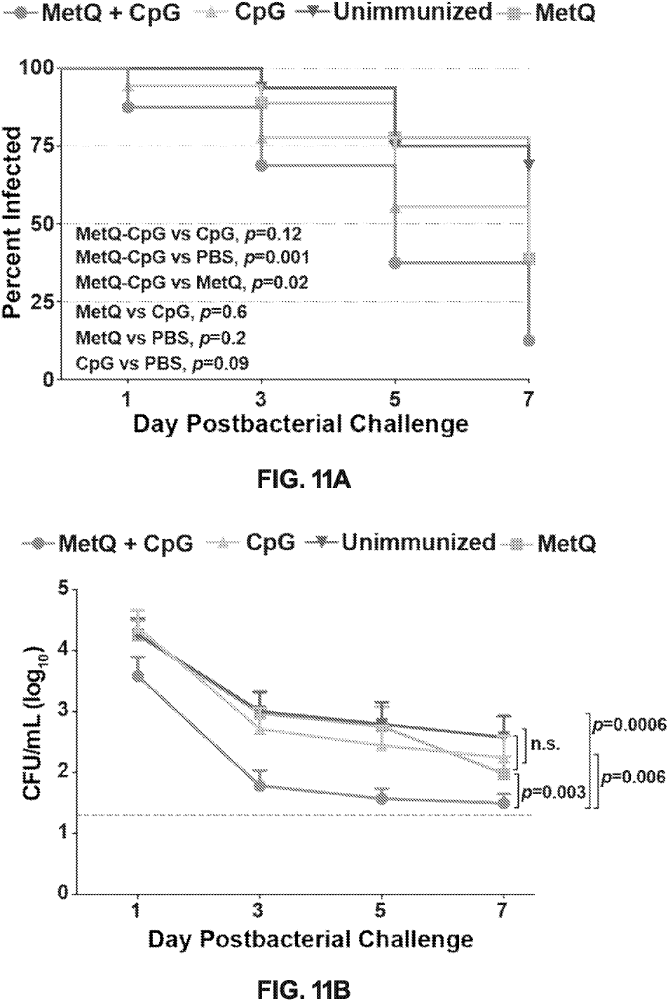

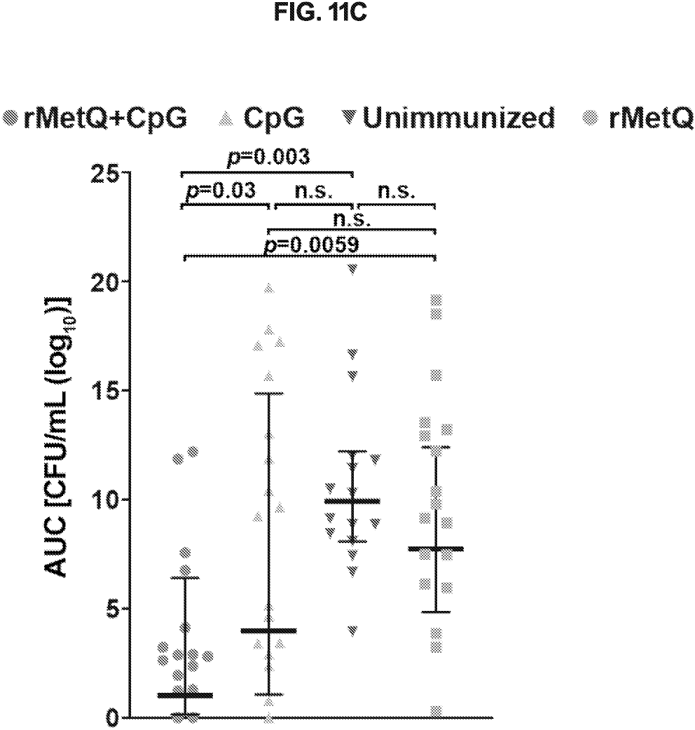

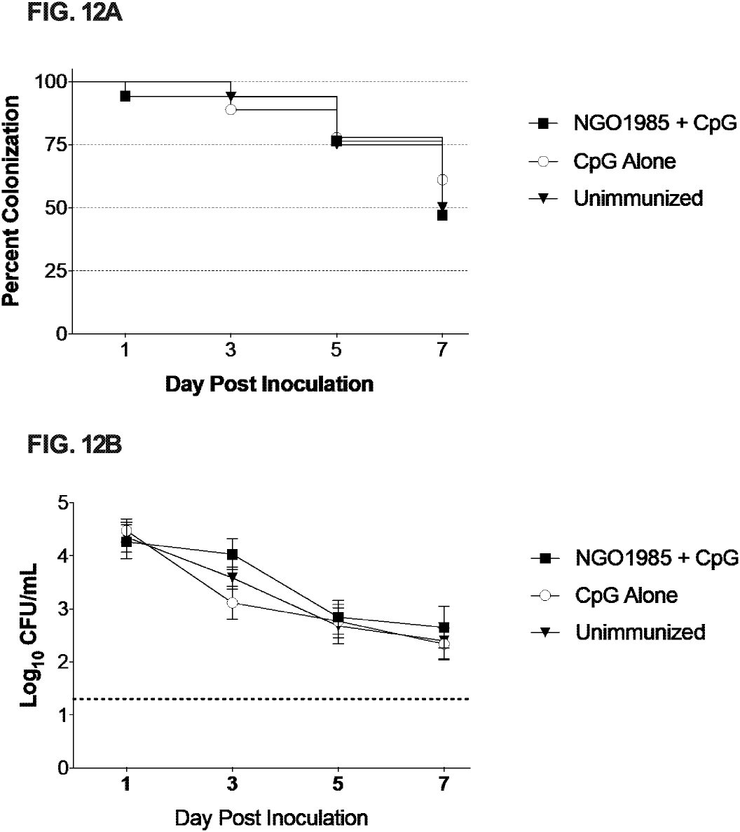

MetQ: A surface protein of Neisseriagonorrhea that, in vivo, is involved in adherence to cervical epithelial cells and is also involved with survival of Neisseriagonorrhea in primary monocytes, primary macrophages, and human serum. Exemplary MetQ sequences found in nature are shown in FIGS. 9A-9B. A recombinant MetQ protein is shown in FIG. 8. MetQ proteins are disclosed in detail below. A “recombinant” MetQ protein is not found in nature.

Neisseriagonorrhoeae. A species of Gram-negative diplococci bacteria isolated by Albert Neisser that causes the sexually transmitted genitourinary infection gonorrhea and forms of gonococcal disease including disseminated gonococcemia, septic arthritis, and gonococcal ophthalmia neonatorum. This bacterium is oxidase positive and aerobic, and it survives within neutrophils. The bacteria can cause infection of the genitals, throat, and eyes.

Pharmaceutically acceptable carriers: The pharmaceutically acceptable carriers of use are conventional. Remington’s Pharmaceutical Sciences, by E. W. Martin, Mack Publishing Co., Easton, PA, 19th Edition, 1995, describes compositions and formulations suitable for pharmaceutical delivery of the disclosed immunogens.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol, or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions (such as immunogenic compositions) to be administered can contain minor amounts of non-toxic auxiliary substances, such as wetting or emulsifying agents, preservatives, and pH buffering agents and the like, for example sodium acetate or sorbitan monolaurate. In particular embodiments, suitable for administration to a subject the carrier may be sterile, and/or suspended or otherwise contained in a unit dosage form containing one or more measured doses of the composition suitable to induce the desired immune response. It may also be accompanied by medications for its use for treatment purposes. The unit dosage form may be, for example, in a sealed vial that contains sterile contents or a syringe for injection into a subject, or lyophilized for subsequent solubilization and administration or in a solid or controlled release dosage.

Polypeptide: Any chain of amino acids, regardless of length or post-translational modification (e.g., glycosylation or phosphorylation). “Polypeptide” applies to amino acid polymers including naturally occurring amino acid polymers and non-naturally occurring amino acid polymer as well as in which one or more amino acid residue is a non-natural amino acid, for example, an artificial chemical mimetic of a corresponding naturally occurring amino acid. A “residue” refers to an amino acid or amino acid mimetic incorporated in a polypeptide by an amide bond or amide bond mimetic. A polypeptide has an amino terminal (N-terminal) end and a carboxy terminal (C-terminal) end. “Polypeptide” is used interchangeably with peptide or protein, and is used herein to refer to a polymer of amino acid residues.

Prime-boost vaccination: An immunotherapy including administration of a first immunogenic composition (the primer vaccine) followed by administration of another immunogenic composition (the booster vaccine) to a subject to induce an immune response. The primer vaccine and/or the booster vaccine are immunogens to which the immune response is directed. The booster vaccine is administered to the subject after the primer vaccine; a suitable time interval between administration of the primer vaccine and the booster vaccine, and examples of such timeframes are disclosed herein. In some embodiments, the primer vaccine, the booster vaccine, or both primer vaccine and the booster vaccine additionally include an adjuvant.

Recombinant: A recombinant nucleic acid molecule or recombinant protein is one that has a sequence that is not naturally occurring, for example, includes one or more substitutions, deletions or insertions, and/or has a sequence that is made by an artificial combination of two otherwise separated segments of sequence. This artificial combination can be accomplished by chemical synthesis or, more commonly, by the artificial manipulation of isolated segments of nucleic acids, for example, by genetic engineering techniques. In several embodiments, a recombinant protein is encoded by a heterologous (for example, recombinant) nucleic acid that has been introduced into a host cell, such as a bacterial or eukaryotic cell, or into the genome of a recombinant virus.

Sequence identity: The similarity between amino acid sequences is expressed in terms of the similarity between the sequences, otherwise referred to as sequence identity. Sequence identity is frequently measured in terms of percentage identity; the higher the percentage, the more similar the two sequences are. Homologs, orthologs, or variants of a polypeptide will possess a relatively high degree of sequence identity when aligned using standard methods.

Methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16: 10881-90, 1988; Huang et al. Computer Appls. In the Biosciences 8, 155-65, 1992; and Pearson et al., Meth. Mol. Bio. 24:307-31, 1994. Altschul et al., J. Mol. Biol. 215:403-10, 1990, presents a detailed consideration of sequence alignment methods and homology calculations.

Variants of a polypeptide are typically characterized by possession of at least about 75%, for example, at least about 80%, 85%, 90%, 91%, 92%, 93%, 94%, 95%, 96%, 97%, 98% or 99% sequence identity counted over the full-length alignment with the amino acid sequence of interest. Proteins with even greater similarity to the reference sequences will show increasing percentage identities when assessed by this method, such as at least 80%, at least 85%, at least 90%, at least 95%, at least 98%, or at least 99% sequence identity. When less than the entire sequence is being compared for sequence identity, homologs and variants will typically possess at least 80% sequence identity over short windows of 10-20 amino acids, and may possess sequence identities of at least 85% or at least 90% or 95% depending on their similarity to the reference sequence. Methods for determining sequence identity over such short windows are available at the NCBI website on the internet.

As used herein, reference to “at least 90% identity” (or similar language) refers to “at least 90%, at least 91%, at least 92%, at least 93%, at least 94%, at least 95%, at least 96%, at least 97%, at least 98%, at least 99%, or even 100% identity” to a specified reference sequence.

Subject: Living multi-cellular vertebrate organisms, a category that includes human and non-human mammals. In an example, a subject is a human. In an additional example, a subject is selected that is in need of inhibiting of a Neisseria infection. For example, the subject is either uninfected and at risk for infection, or is infected in need of treatment.

Under conditions sufficient for: A phrase that is used to describe any environment that permits a desired activity.

Vaccine: A preparation of immunogenic material capable of stimulating an immune response, administered for the prevention, amelioration, or treatment of infectious or other types of disease. The immunogenic material may include attenuated or killed microorganisms (such as bacteria or viruses), or antigenic proteins, peptides, or DNA derived from them. A vaccine may include a disclosed immunogen, such as a MetQ protein, and an adjuvant, such as a CpG ODN. Vaccines can elicit both prophylactic (preventative or protective) and therapeutic responses. Methods of administration vary according to the vaccine, but may include inoculation, ingestion, inhalation, or other forms of administration. In one specific, non-limiting example, a vaccine prevents and/or reduces the severity of the symptoms associated with a Neisseria gonorrhoeae infection and/or decreases the viral load compared to a control.

All publications, patent applications, patents, and other references mentioned herein are incorporated by reference in their entirety. In case of conflict, the present specification, including explanations of terms, will control. In addition, the materials, methods, and examples are illustrative only and not intended to be limiting.

### MetQ

MetQ is a surface antigen of Neisseriagonorrhea. A sequence analysis of MetQ is provided in Semchenko et al., Infection and Immunity 85: e00898-16, February 2017, incorporated herein by reference. The naturally occurring protein is generally 288 amino acids in length with an N-terminal methionine. In vivo, the N-terminal methionine is removed with the signal peptide during protein transport across the bacterial membrane and includes a methionine binding component of an ATP-binding cassette transport system. The first cytosine can be modified and MetQ can be anchored to the bacterial surface. In nature, the Neisseriagonorrhea MetQ protein is 98% identical to the MetQ protein of Neisseriameningitidis and 38% identical to the MetQ protein of E.coli. In vivo, the MetQ protein is localized on the surface of Neisseriagonorrhea.

MetQ is involved in adherence to cervical epithelial cells and is also involved with survival of Neisseriagonorrhea in primary monocytes, primary macrophages, and human serum. Anti-MetQ antibodies have been shown to be bactericidal and can reduce adherence to cervical epithelial cells (Semchenko et al., supra, 2017).

A Neisseriagonorrhea MetQ protein reference sequence (SEQ ID NO: 1) is provided below. MetQ protein residue numbering used herein is made with reference to SEQ ID NO: 1. With reference to position number, the N-terminal amino acid is position one, and the remaining positions are the amino acids, as numbered sequentially.

MKTFFKTLSAAALALILAACGGQKDSAPAASAAAPSADNGAAKKEIVFGTTVGDFGDMVK 60
EQIQAELEKKGYTVKLVEFTDYVRPNLALAEGELDINVFQHKPYLDDFKKEHNLDITEAF 120
QVPTAPLGLYPGKLKSLEEVKDGSTVSAPNDPSNFARALVMLNELGWIKLKDGINPLTAS 180
KADIAENLKNIKIVELEAAQLPRSRADVDFAVVNGNYAISSGMKLTEALFQEPSFAYVNW 240
SAVKTADKDSQWLKDVTEAYNSDAFKAYAHKRFEGYKYPAAWNEGAAK 289

In this sequence, positions 1-19 are a signal sequence (underlined above in SEQ ID NO: 1). In some embodiments, the MetQ protein does not include a signal sequence, such as positions 1-19. In further embodiments, the MetQ protein also does not include positions 20-23, such as the sequence CGGQ (amino acids 20-23 of SEQ ID NO: 1) shown in SEQ ID NO: 1 above. Thus, in some embodiments, positions 1-23 are not present. In some embodiments, the MetQ protein includes positions 24-289 of SEQ ID NO: 1. In a specific non-limiting example, the MetQ protein can include amino acids 24-289 of SEQ ID NO: 1.

In some embodiments, and MetQ protein of use in the disclosed methods is at includes a sequence least 95%, 96%, 97%, 98%, 99% identical to amino acids 24-289 of SEQ ID NO: 1. The MetQ protein can include at most 15, 14, 13, 12, 11, 10, 9, 8, 7, 6, 5, 4, 3, 2 or 1 conservative substitutions in amino acids 24-289 of SEQ ID NO: 1. The MetQ protein can be naturally occurring or recombinant.

Additional MetQ proteins are shown in FIGS. 8 and 9. In some embodiments, the MetQ protein includes one of more of the following mutations: A27V, A33S, S36A, A42V, A65P, A65V, A119V, A158V, N163D, S220G, D263N, A259V, R272C, Y278S, and N150D. The MetQ protein can include, 1, 2,3, 4, 5, 6, 7, 8, 9, 10, 11, 12, 13, 14 or 15 of these mutations. The MetQ protein can include a deletion of at most 5 amino acids, such as 1, 2, 3, 4, or 5 amino acids. In some embodiments, the MetQ protein can include a deletion of up to 3 amino acids, such as 1, 2 or 3 amino acids. In more embodiments, the MetQ protein can include a deletion of an amino acids, such as, but not limited to a deletion of amino acid 35. These deletions can be combined with one of more of the following mutations: A27V, A33S, S36A, A42V, A65P, A65V, A119V, A158V, N163D, S220G, D263N, A259V, R272C, Y278S, and N150D. In some embodiments, a deletion of amino acid 35 is combined with one or more of these mutations. Exemplary combinations are shown in FIGS. 9A-9B.

In some embodiments, the MetQ protein is recombinant. In some embodiments, the MetQ protein includes the amino acid sequence GAME (SEQ ID NO: 2). In some embodiments, SEQ ID NO: 2 is at the amino terminus of the protein. In some non-limiting examples, SEQ ID NO: 2 is immediately followed by position 29 of SEQ ID NO: 1.

In other embodiments, additional spacers, heterologous to MetQ, such as of 1, 2, 3, 4, 5, or 6 amino acids can be included. In more embodiments, such as spacer is not included.

In further embodiments, the MetQ protein includes the amino acid sequence KLAAA (SEQ ID NO: 3). In some embodiments, SEQ ID NO: 3 is at the carboxy-terminus of the MetQ protein. In some non-limiting examples, position 289 of SEQ ID NO: 1 is immediately following by SEQ ID NO: 3.

In a further embodiment, the MetQ protein includes both SEQ ID NO: 2 and SEQ ID NO: 3. In some embodiments, SEQ ID NO: 2 is at the amino terminus and SEQ ID NO: 3 is at the carboxy terminus of the MetQ protein. In a further embodiment, the MetQ protein includes SEQ ID NO: 2, positions 24-289 of SEQ ID NO: 1 and SEQ ID NO: 3, in amino to carboxy terminus order. In some embodiments, additional amino acids are not included.

An exemplary recombinant MetQ protein of use is disclosed below (SEQ ID NO: 4):

GAMEKDSAPAASAAAPSADNGAAKKEIVFGTTVGDFGDMVKEQIQAELEK
KGYTVKLVEFTDYVRPNLALAEGELDINVFQHKPYLDDFKKEHNLDITEA
FQVPTAPLGLYPGKLKSLEEVKDGSTVSAPNDPSNFARALVMLNELGWIK
LKDGINPLTASKADIAENLKNIKIVELEAAQLPRSRADVDFAVVNGNYAI
SSGMKLTEALFQEPSFAYVNWSAVKTADKDSQWLKDVTEAYNSDAFKAYA
HKRFEGYKYPAAWNEGAAKKLAAA

In some embodiments, and MetQ protein of use in the disclosed methods is at least 95%, 96%, 97%, 98%, 99% identical to SEQ ID NO: 4. The MetQ protein can include at most 15, 14, 13, 12, 11, 10, 9, 8, 7, 6, 5, 4, 3, 2 or 1 conservative substitutions in SEQ ID NO: 4. In further embodiments, the MetQ protein includes the amino acid sequence GAME (SEQ ID NO: 2) at the 5′ end and/or the amino acid sequence KLAAA (SEQ ID NO: 3) at the 3-end. In specific non-limiting examples, both GAME (SEQ ID NO: 2) and/or KLAAA (SEQ ID NO: 3) are included in the MetQ protein.

In some embodiments, the MetQ protein includes SEQ ID NO: 4 with one of more of the following mutations: A27V, A33S, S36A, A42V, A65P, A65V, A119V, A158V, N163D, S220G, D263N, A259V, R272C, Y278S, and N150D. This residue numbering is with reference to SEQ ID NO: 1. The MetQ protein can include, 1, 2,3, 4, 5, 6, 7, 8, 9, 10, 11, 12, 13, 14 or 15 of these mutations. Exemplary combinations of mutations are listed in the table shown in FIGS. 9A-9B.

The MetQ protein can include a deletion of at most 5 amino acids, such as 1, 2, 3, 4, or 5 amino acids. In some embodiments, the MetQ protein can include a deletion of up to 3 amino acids, such as 1, 2 or 3 amino acids. In more embodiments, the MetQ protein can include a deletion of an amino acids, such as, but not limited to a deletion of amino acid 35. These deletions can be combined with one of more of the following mutations: A27V, A33S, S36A, A42V, A65P, A65V, A119V, A158V, N163D, S220G, D263N, A259V, R272C, Y278S, and N150D. In some embodiments, a deletion of amino acid 35 is combined with one or more of these mutations. An exemplary combination is shown in FIGS. 9A-9B.

The disclosed MetQ proteins can be prepared using recombinant methods, such as expression in host cells. Exemplary nucleic acid molecules can be prepared by cloning techniques. Examples of appropriate cloning and sequencing techniques, and instructions sufficient to direct persons of skill through many cloning exercises are known (see, e.g., Sambrook et al. (Molecular Cloning: A Laboratory Manual, 4^{th} ed, Cold Spring Harbor, New York, 2012) and Ausubel et al. (In Current Protocols in Molecular Biology, John Wiley & Sons, New York, through supplement 104, 2013).

Nucleic acid molecules can also be prepared by amplification methods. Amplification methods include polymerase chain reaction (PCR), the ligase chain reaction (LCR), the transcription-based amplification system (TAS), the self-sustained sequence replication system (3SR). A wide variety of cloning methods, host cells, and in vitro amplification methodologies are well known to persons of skill.

The polynucleotides encoding the MetQ protein can include a recombinant DNA which is incorporated into a vector (such as an expression vector) into an autonomously replicating plasmid or virus or into the genomic DNA of a prokaryote or eukaryote, or which exists as a separate molecule (such as a cDNA) independent of other sequences. The nucleotides can be ribonucleotides, deoxyribonucleotides, or modified forms of either nucleotide. The term includes single and double forms of DNA.

Polynucleotide sequences encoding a MetQ protein can be operatively linked to expression control sequences. An expression control sequence operatively linked to a coding sequence is ligated such that expression of the coding sequence is achieved under conditions compatible with the expression control sequences. The expression control sequences include, but are not limited to, appropriate promoters, enhancers, transcription terminators, a start codon (i.e., ATG) in front of a protein-encoding gene, splicing signal for introns, maintenance of the correct reading frame of that gene to permit proper translation of mRNA, and stop codons.

DNA sequences encoding the MetQ protein can be expressed in vitro by DNA transfer into a suitable host cell. The cell may be prokaryotic or eukaryotic. The term also includes any progeny of the subject host cell. It is understood that all progeny may not be identical to the parental cell since there may be mutations that occur during replication. Methods of stable transfer, meaning that the foreign DNA is continuously maintained in the host, are known in the art.

Hosts can include microbial, yeast, insect and mammalian organisms. Methods of expressing DNA sequences having eukaryotic or viral sequences in prokaryotes are well known in the art. Non-limiting examples of suitable host cells include bacteria, archea, insect, fungi (for example, yeast), plant, and animal cells (for example, mammalian cells, such as human). Exemplary cells of use include Escherichiacoli, Bacillussubtilis, Saccharomycescerevisiae, Salmonellatyphimurium, SF9 cells, C129 cells, 293 cells, Neurospora, and immortalized mammalian myeloid and lymphoid cell lines. Techniques for the propagation of mammalian cells in culture are well-known (see, e.g., Helgason and Miller (Eds.), 2012, Basic Cell Culture Protocols (Methods in Molecular Biology), 4^{th} Ed., Humana Press). Examples of commonly used mammalian host cell lines are VERO and HeLa cells, CHO cells, and WI38, BHK, and COS cell lines, although cell lines may be used, such as cells designed to provide higher expression, desirable glycosylation patterns, or other features. In some embodiments, the host cells include HEK293 cells or derivatives thereof, such as GnTI^{-/-} cells (ATCCOO No. CRL-3022), or HEK-293F cells.

Transformation of a host cell with recombinant DNA can be carried out by conventional techniques. In some embodiments where the host is prokaryotic, such as, but not limited to, E.coli, competent cells which are capable of DNA uptake can be prepared from cells harvested after exponential growth phase and subsequently treated by the CaCl₂ method. Alternatively, MgCl₂ or RbCl can be used. Transformation can also be performed after forming a protoplast of the host cell if desired, or by electroporation.

When the host is a eukaryote, such methods of transfection of DNA as calcium phosphate coprecipitates, conventional mechanical procedures such as microinjection, electroporation, insertion of a plasmid encased in liposomes, or viral vectors can be used. Eukaryotic cells can also be co-transformed with polynucleotide sequences encoding a disclosed antigen, and a second foreign DNA molecule encoding a selectable phenotype, such as the herpes simplex thymidine kinase gene. Another method is to use a eukaryotic viral vector, such as simian virus 40 (SV40) or bovine papilloma virus, to transiently infect or transform eukaryotic cells and express the protein (see for example, Viral Expression Vectors, Springer press, Muzyczka ed., 2011). Appropriate expression systems such as plasmids and vectors of use in producing proteins in cells including higher eukaryotic cells such as the COS, CHO, HeLa and myeloma cell lines.

A nucleic acid molecule encoding a protomer of a MetQ protein can be included in a viral vector, for example for expression of the protomer to produce the corresponding MetQ protein in a host cell, or for immunization of a subject as disclosed herein. In some embodiments, the viral vectors are administered to a subject as part of a prime-boost vaccination. Typically, such viral vectors include a nucleic acid molecule encoding a MetQ protein. In several embodiments, the viral vectors are included in a vaccine, such as a primer vaccine or a booster vaccine for use in a prime-boost vaccination.

In some examples, the viral vector encoding the MetQ protein can be replication-competent. For example, the viral vector can have a mutation (e.g., insertion of nucleic acid encoding the protomer) in the viral genome that attenuates, but does not completely block viral replication in host cells.

In several embodiments, the viral vector encoding the MetQ protein is a viral vector that can be delivered via the respiratory tract. For example, a hPIV vector, such as bovine parainfluenza virus (BPIV) vector (e.g., a BPIV1, BPIV2, or BPIV3 vector) or human hPIV vector, a metapneumovirus (MPV) vector, a Sendia virus vector, or a measles virus vector, is used to express a disclosed antigen.

Additional viral vectors are also available for expression of the disclosed antigens, including polyoma, i.e., SV40 (Madzak et al., 1992, J. Gen. Virol., 73:15331536), adenovirus (Berkner, 1992, Cur. Top. Microbiol. Immunol., 158:39-6; Berliner et al., 1988, Bio Techniques, 6:616-629; Gorziglia et al., 1992, J. Virol., 66:4407-4412; Quantin et al., 1992, Proc. Natl. Acad. Sci. USA, 89:2581-2584; Rosenfeld et al., 1992, Cell, 68:143-155; Wilkinson et al., 1992, Nucl. Acids Res., 20:2233-2239; Stratford-Perricaudet et al., 1990, Hum. Gene Ther., 1:241-256), vaccinia virus (Mackett et al., 1992, Biotechnology, 24:495-499), adeno-associated virus (Muzyczka, 1992, Curr. Top. Microbiol. Immunol., 158:91-123; On et al., 1990, Gene, 89:279-282), herpes viruses including HSV and EBV and CMV (Margolskee, 1992, Curr. Top. Microbiol. Immunol., 158:67-90; Johnson et al., 1992, J. Virol., 66:29522965; Fink et al., 1992, Hum. Gene Ther. 3: 11-19; Breakfield et al., 1987, Mol. Neurobiol., 1:337-371; Fresse etal., 1990, Biochem. Pharmacol., 40:2189-2199), Sindbis viruses (H. Herweijer et al., 1995, Human Gene Therapy 6:1161-1167; U.S. Pat. Nos. 5,091,309 and 5,2217,879), alphaviruses (S. Schlesinger, 1993, Trends Biotechnol. 11:18-22; I. Frolov et al., 1996, Proc. Natl. Acad. Sci. USA 93:11371-11377) and retroviruses of avian (Brandyopadhyay et al., 1984, Mol. Cell Biol., 4:749-754; Petropouplos et al., 1992, J. Virol., 66:3391-3397), murine (Miller, 1992, Curr. Top. Microbiol. Immunol., 158:1-24; Miller et al., 1985, Mol. Cell Biol., 5:431-437; Sorge et al., 1984,Mol. Cell Biol., 4: 1730-1737; Mann et al., 1985, J. Virol., 54:401-407), and human origin (Page et al., 1990, J. Virol., 64:5370-5276; Buchschalcher et al., 1992, J. Virol., 66:2731-2739). Baculovirus (Autographa californica multinuclear polyhedrosis virus; AcMNPV) vectors are also known in the art, and may be obtained from commercial sources (such as PharMingen, San Diego, Calif.; Protein Sciences Corp., Meriden, Conn.; Stratagene, La Jolla, Calif.).

### B. K-Type ODNs (Also Referred to as CpG-B)

K-type ODNs are linear, single-stranded polynucleotides that include unmethylated CpG motifs. K ODNs trigger a NF-κB-dependent proinflammatory response characterized by the production of IL-6 and TNF-α by pDCs. K-type ODNs also stimulate B cells to proliferate and secrete IgM. In contrast, D-type ODNs (also referred to as CpG-A) form complex stem-loop structures and have a poly-G tail that leads them to form G-tetrads. D-type ODNs stimulate pDCs to produce IFN-α/β rather than TNF-α, an effect that is amplified though an autocrine feedback loop. Both K-type and D-type ODNs signal through Toll-like receptor (TLR)9.

Briefly, the K-type CpG ODN nucleic acid sequences useful in the methods disclosed herein are represented by the formula:

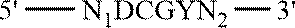

wherein at least one nucleotide separates consecutive CpGs; D is adenine, guanine, or thymidine; Y is cytosine or thymine, N is any nucleotide and N₁ + N₂ is from about 0-26 bases. In one embodiment, N₁ and N₂ do not contain a CCGG quadmer or more than one CGG trimer; and the nucleic acid sequence is from about 8-30 bases in length, such as about 10 to 30 nucleotides in length. However, nucleic acids of any size (even many kb long) can be used in the methods disclosed herein if CpGs are present. In one embodiment, synthetic oligonucleotides of use do not include a CCGG quadmer or more than one CCG or CGG trimer at or near the 5′ or 3′ terminals and/or the consensus mitogenic CpG motif is not a palindrome. A “palindromic sequence” or “palindrome” means an inverted repeat (i.e., a sequence such as ABCDEE′D′C′B′A′, in which A and A′ are bases capable of forming the usual Watson-Crick base pairs).

In another embodiment, the methods include the use of an ODN which contains a CpG motif represented by the formula:

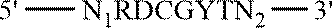

wherein at least one nucleotide separates consecutive CpGs; RD is selected from the group consisting of GpT, GpG, GpA, ApT and ApA; YT is selected from the group consisting of TpT or CpT; N is any nucleotide and N₁ + N₂ is from about 0-26 bases, such that the ODN is about 8 to 30 nucleotides in length.

In several embodiments, the methods disclosed herein include the use of an effective amount of at least one K-type CpG ODN, wherein the K-type CpG ODNs include an unmethylated CpG motif that has a sequence represented by the formula:

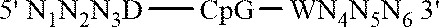

wherein the central CpG motif is unmethylated, D is T, G or A, W is A or T, and N₁, N₂, N₃, N₄, N₅, and N₆ are any nucleotides. In one embodiment, D is a T. The K ODN(s) can be 10 to 30 nucleotides in length. A K ODN can include multiple CpG motifs. In some embodiments, at least one nucleotide separates consecutive CpGs; N₃D is selected from the group consisting of GpT, GpG, GpA, ApT and ApA; WN₄ is selected from the group consisting of TpT or CpT; N is any nucleotide and N₁ + N₂ is from about 0-26 bases.

In one embodiment, N₁, and N₂ do not contain a CCGG quadmer or more than one CCG or CGG trimer. CpG ODN are also in the range of 8 to 50 bases in length, such as 8 to 30 bases in length, but may be of any size (even many kb long) if sufficient motifs are present. In several examples, the K-type CpG ODN is 10 to 20 nucleotides in length, such as 12 to 18 nucleotides in length. In one embodiment, synthetic ODNs of this formula do not include a CCGG quadmer or more than one CCG or CGG trimer at or near the 5′ and/or 3′ terminals and/or the consensus CpG motif is not a palindrome. Other CpG ODNs can be assayed for efficacy using methods described herein. It should be noted that exemplary K-type CpG ODNs are known in the art, and have been fully described, for example in PCT Publication No. WO 98/18810A1, and WO 01/22972, which are incorporated herein by reference. The K type OD can be stabilized.

Exemplary K ODN are listed below

| | | |
|---|---|---|
| K X | ATAATCGACGTTCAAGCAAG | (SEQ ID NO: 8) |
| K22 | CTCGAGCGTTCTC | (SEQ ID NO: 9) |
| K21 | TCTCGAGCGTTCTC | (SEQ ID NO: 10) |
| K82 | ACTCTGGAGCGTTCTC | (SEQ ID NO: 11) |
| K30 | TGCAGCGTTCTC | (SEQ ID NO: 12) |
| k31 | TCGAGGCTTCTC | (SEQ ID NO: 13) |
| K39 | GTCGGCGTTGAC | (SEQ ID NO: 14) |
| K16 | TCGACTCTCGAGCGTTCTC | (SEQ ID NO: 15) |
| K3 | ATCGACTCTCGAGCGTTCTC | (SEQ ID NO: 16) |
| k23 | TCGAGCGTTCTC | (SEQ ID NO: 17) |
| K40 | GTCGGCGTCGAC | (SEQ ID NO: 18) |
| K34 | GTCGACGTTGAC | (SEQ ID NO: 19) |
| K83 | ACTCTCGAGGGTTCTC | (SEQ ID NO: 20) |
| K19 | ACTCTCGAGCGTTCTC | (SEQ ID NO: 21) |
| K73 | GTCGTCGATGAC | (SEQ ID NO:22) |
| K46 | GTCGACGCTGAC | (SEQ ID NO:23) |
| K47 | GTCGACGTCGAC | (SEQ ID NO:24) |
| K72 | GTCATCGATGCA | (SEQ ID NO:25) |
| K37 | GTCAGCGTCGAC | (SEQ ID NO:26) |
| k25 | TCGAGCGTTCT | (SEQ ID NO:27) |
| K82 | ACTCTGGAGCGTTCTC | (SEQ ID NO: 28) |
| K83 | ACTCTCGAGGGTTCTC | (SEQ ID NO:29) |
| K84 | ACTCTCGAGCGTTCTA | (SEQ ID NO: 30) |
| K85 | CATCTCGAGCGTTCTC | (SEQ ID NO: 31) |
| K89 | ACTCTTTCGTTCTC | (SEQ ID NO: 32) |
| K109 | TCGAGCGTTCT | (SEQ ID NO: 33) |
| K123 | TCGTTCGTTCTC | (SEQ ID NO: 34) |
| K1555 | GCTAGACGTTAGCGT | (SEQ ID NO: 35) |
| K110 | TCGAGGCTTCTC | (SEQ ID NO: 36) |
| CpG10103 | TCGTCGTTTTACGGCGCCGTGCCG | (SEQ ID NO: 37) |
| CpG7909 | TCGTCGTTTTGTCGTTTTGTCGTT | (SEQ ID NO: 38) |
| ODN 1826 | TCCATGACGTTCCTGACGTT | (SEQ ID NO: 39) |
| ODN 2216 | ggGGGACGATCGTCgggggg* | (SEQ ID NO: 40) |
| ODN 2336 | gggGACGACGTCGTGgggggg* | (SEQ ID NO: 41) |
| ODN1018 | TGACTGTGAACGTTCGAGATGA | (SEQ ID NO: 42) |

*bases shown in capital letters are phophodiester, and those in lower case are phosphorothioate

A single K-type CpG ODN can be used in the methods disclosed herein. In some embodiments, the K-type CpG ODN comprises or consists of the nucleic acid sequence set forth as one of SEQ ID NO: 3-34. The K-type CpG ODN can be any ODN listed above, including but not limited to ODN 1826, ODN 2216, ODN 2336, CpG7909, K155 or K3. In some embodiments, the K-type CpG ODN is ODN 1826. In other embodiments, the K-type CpG ODN is ODN 2216. In more embodiments, the K-type CpG ODN is ODN 2336. In further embodiments, the K-type CpG ODN is ODN CpG7909. In some embodiments, the K-type CpG ODN is K155. In more embodiments, the K-type CpG ODN is K3. In further embodiments, the K-type CpG ODN is ODN1018. The ODN can be any one of SEQ ID NOs: 8-42.

However, it is also possible to use mixtures of K-type CpG ODNs having more than one K-type CpG ODN and an imidazoquinoline compound. Exemplary combinations that can be used include 1) K3, K19, K110; 2) K19, K23, K123; K3, 3) K110, K123;4) K3, K23, K123; 5) K3, K19, K123; and 6) K19, K110, K123. Additional exemplary combinations include at least two different K-type CpG ODNs, wherein one of the K-type CpG ODNs is K1555, and/or wherein one of the K-type CpG ODNs is K3. In some embodiments, one of the ODNs is ODN 1826, ODN 2216, or ODN 2236. In some embodiments, one of the ODNs is CpG 7909. In other embodiments, one of the ODNs is ODN1018.

For use in the methods disclosed herein, ODNs can be synthesized de novo using any of a number of procedures well known in the art. For example, the b-cyanoethylphosphoramidite method (Beaucage et al., Tet. Let. 22: 1859, 1981) or the nucleoside H-phosphonate method (Garegg et al., Tet. Let. 27:4051, 1986; Froehleret al., Nucl. Acid Res. 14:5399, 1986; Garegg et al., Tet. Let. 27:4055, 1986; Gaffney et al., Tet. Let. 29:2619, 1988) can be utilized. These chemistries can be performed by a variety of automated oligonucleotide synthesizers available in the market.

Alternatively, ODNs can be produced on a large scale in plasmids, (see Sambrook, et al., Molecular Cloning: A Laboratory Manual, Cold Spring Harbor Laboratory Press, New York, 1989) which after being administered to a subject are degraded into oligonucleotides. ODNs can be prepared from existing nucleic acid sequences (e.g., genomic or cDNA) using known techniques, such as those employing restriction enzymes, exonucleases or endonucleases (see PCT Application No. PCT/US98/03678).

As noted above, for use in vivo, nucleic acids can be utilized that are relatively resistant to degradation (such as by endo-and exo-nucleases). Nucleic acid stabilization can be accomplished via phosphate backbone modifications of CpG ODNs. In one embodiment, a stabilized nucleic acid has at least a partial phosphorothioate modified backbone. Phosphorothioates may be synthesized using automated techniques employing either phosphoramidate or H-phosphonate chemistries. Aryl-and alkyl-phosphonates can be made (e.g., as described in U.S. Pat. No. 4,469,863) and alkylphosphotriesters (in which the charged oxygen moiety isalkylated, as described in U.S. Pat. No. 5,023,243 and European Patent No. 092,574), and can be prepared by automated solid phase synthesis using commercially available reagents.

In one embodiment, the phosphate backbone modification occurs at the 5′ end of the ODN. One specific, non-limiting example of a phosphate backbone modification is at the first two nucleotides of the 5′ end of the nucleic acid. In another embodiment, the phosphate backbone modification occurs at the 3′ end of the nucleic acid. One specific, non-limiting example of a phosphate backbone modification is at the last five nucleotides of the 3′ end of the nucleic acid.

Methods for making other DNA backbone modifications and substitutions have been described (Uhlmann et al., Chem. Rev. 90:544, 1990; Goodchild, Bioconjugate Chem. 1:1, 1990). 2’-O-methyl nucleic acids with CpG motifs also cause angiogenesis, as do ethoxy-modified CpG nucleic acids. In fact, no backbone modifications have been found that completely abolish the CpG effect, although it is greatly reduced by replacing the C with a 5-methyl C.

For administration in vivo, nucleic acids (including immunosuppressive ODNs) can be associated with a molecule that results in higher affinity binding to target cell (such as an epithelial cell) surfaces and/or increased cellular uptake by target cells to form a “nucleic acid delivery complex.” Nucleic acids can be ionically or covalently associated with appropriate molecules using techniques which are well known in the art (see below). Nucleic acids can alternatively be encapsulated in liposomes or virosomes using well-known techniques.

An CpG ODN can be associated with (for example, ionically or covalently bound to, or encapsulated within) a targeting moiety. Targeting moieties include any molecule that results in higher affinity binding to a target cell. For example, for an immunostimulatory CpG ODN (D-type or K-type), a targeting molecule can target the ODN to cells that express TLR9, including B cells and plasmacytoid dendritic cells.

A variety of coupling or cross-linking agents can be used to form the delivery complex, such as protein A, carbodiamide, and N-succinimidyl (2-pyridyldithio) propionate (SPDP). Examples of delivery complexes include ODNs associated with a sterol (such as cholesterol), a lipid (such as a cationic lipid, virosome or liposome), and a target cell specific binding agent (such as a ligand recognized by target cell specific receptor). In one embodiment, the complexes are sufficiently stable in vivo to prevent significant uncoupling prior to internalization by the target cell. However, these complexes can be cleavable under appropriate circumstances such that the ODN can be released in a functional form (see, for example, PCT Application No. WO 00/61151).

### Immunogenic Compositions and Methods of Use

The disclosed methods include the use of a MetQ protein and a K-type CpG ODN. The MetQ protein can be a recombinant MetQ protein. In some embodiments, a single pharmaceutical composition is administered to a subject that includes an effective amount of a CpG ODN and the MetQ protein. The MetQ protein and the K-type CpG ODN can be administered as separate compositions, provided the K-type CpG ODN increases the immune response to the MetQ protein. In some embodiments, the MetQ protein and the K-type CpG ODN are administered simultaneously, or within minutes of each other, such as within 5, 10, 15 or 20 minutes.

In some embodiments, about 1 to about 100 µg/gm CpG ODN, such as about 5 to about 50 µg/gm, such as about 50 µg/gm of the CpG ODN are administered to the subject. In other embodiments, about 1 to about 100 mg/kg, such as about 5 to about 50 mg/kg, such as about 10 mg/kg, of the CpG are administered to the subject. In additional embodiments, the effective amount of the CpG ODN can vary from about 0.01 µg/kg to about 1 g/kg, such as about 1 µg/kg to about 5 mg/kg, or about 5 µgkg to about 1 mg/kg. The exact dose is readily determined by one of skill in the art based on factors such as the age, weight, sex and physiological condition of the subject.

One MetQ:CpG ratio is 1:67 (wt/wt). However, other ratios can be used, such as 1:20, 1:30, 1:40, 1:50, 1:33, 1;60, 1:65, 1:70, 1:75, 1:80 or 1:85).

The amount of MeQ protein included in the immunogenic composition is sufficient to elicit an immune response, such as a humoral immune response and/or a cellular immune response, in the subject. In some embodiments, amounts for the immunization generally range from about 0.001 mg to about 1.0 mg per 70 kilogram subject, more commonly from about 0.001 mg to about 0.2 mg per 70 kilogram subject. Dosages from 0.001 up to about 10 mg per subject per day may be used, particularly when the antigen is administered to a secluded site and not into the bloodstream, such as into a body cavity or into a lumen of an organ. Substantially higher dosages (e.g. 10 to 100 mg or more) are possible in oral, nasal, or topical administration.

In other embodiments, each human dose will comprise 1-1000 µg of MetQ protein, such as from about 1 µg to about 100 µg, for example, from about 1 µg to about 50 µg, such as about 1 µg, about 2 µg, about 5 µg, about 10 µg, about 15 µg, about 20 µg, about 25 µg, about 30 µg, about 40 µg, or about 50 µg MetQ protein. The amount utilized is selected based on the subject, such as based on their age, weight and other clinical parameters.

An optimal amount for a particular composition can be ascertained by standard studies, such as using observation of antibody titers and other immune responses. Determination of effective dosages is typically based on animal model studies followed up by human clinical trials and is guided by administration protocols that significantly reduce the occurrence or severity of targeted disease symptoms or conditions in the subject, or that induce a desired response in the subject (such as an antibody response). Suitable models in this regard include, for example, murine, rat, porcine, feline, ferret, non-human primate, and other accepted animal model subjects known in the art. Alternatively, effective dosages can be determined using in vitro models (for example, immunologic and histopathologic assays). Using such models, only ordinary calculations and adjustments are required to determine an appropriate concentration and dose to administer an effective amount of the composition (for example, amounts that are effective to elicit a desired immune response or alleviate one or more symptoms of disease). In alternative embodiments, an effective amount or effective dose of the composition may simply inhibit or enhance one or more selected biological activities correlated with a gonorrhea or condition, as set forth herein.

In some embodiments, the MetQ protein and the CpG ODN can be administered via the intramuscular, intraperitoneal, intradermal, or subcutaneous routes; or via mucosal administration to the oral/alimentary, respiratory (e.g., intranasal administration), genitourinary tracts. Although the immunogenic composition can be administered as a single dose, components thereof (the MetQ protein and the CpG ODN) can also be co-administered together at the same time or at different times. In addition to a single route of administration, two or more different routes of administration can be used.

Methods are disclosed herein for inducing an immune response to Neisseriagonorrhoeae in a mammalian subject using any of the disclosed immunogenic compositions. The immune response can be a protective immune response or a therapeutic immune response. The subject can be a human or veterinary subject. The subject can be an adult or a juvenile subject. The subject can be in infant. In some embodiments, the subject is a human child of 10, 11, 12, 13, 14, 15, 16, or 17 years of age. The subject can be an adult, such as a human subject 18 or more years of age. The subject can be an infant, such as a human subject that is less than one year of age. The subject can be a newborn, such as a human subject less than one month of age.

The subject can be a male or a female. In some embodiments, the subject is a female, and an immune response is produced in the vaginal mucosa.

Immunogenic compositions are provided that include a K-type CpG ODN, a MetQ protein, and a pharmaceutically acceptable carrier. The MetQ protein can be a recombinant MetQ protein, as disclosed above. In specific non-limiting example, the MetQ protein comprises SEQ ID NO: 4. On other specific non-limiting examples, the CpG ODN is ODN1826 or CpG7909.

The immunogenic compositions also can also include other agents, such as binders. Binders include, but are not limited to, carboxymethylcellulose, ethyl cellulose, microcrystalline cellulose, or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin, a flavoring agent such as peppermint, methyl salicylate or orange flavoring, and a coloring agent. The compositions can also include gum arabic, syrup, lanolin, starch, etc., that forms a vehicle for delivery. Included are substances that, in the presence of sufficient liquid, impart to a composition the adhesive quality needed for the preparation of pills or tablets.

Exemplary “pharmaceutically acceptable carriers” include liquid carriers (such as water, saline, culture medium, aqueous dextrose, and glycols) and solid carriers (such as carbohydrates exemplified by starch, glucose, lactose, sucrose, and dextrans, anti-oxidants exemplified by ascorbic acid and glutathione, and hydrolyzed proteins). Exemplary diluents include water, physiological saline solution, human serum albumin, oils, polyethylene glycols, glycerine, propylene glycol, or other synthetic solvents. The compositions can also include antibacterial agents such as benzyl alcohol, antioxidants such as ascorbic acid or sodium bisulphite, chelating agents such as ethylene diamine-tetra-acetic acid, buffers such as acetates, citrates or phosphates, and agents for adjusting the osmolarity, such as sodium chloride or dextrose.

Immunogenic compositions can be lyophilized or be in aqueous form, e.g., solutions or suspensions. Liquid formulations allow the compositions to be administered directly from their packaged form, without the need for reconstitution in an aqueous medium. Compositions can be presented in vials, or they can be presented in ready-filled syringes. The syringes can be supplied with or without needles. A syringe will include a single dose of the composition, whereas a vial can include a single dose or multiple doses (e.g. 2, 3, 4, 5, 6, 7, 8, 9, or 10 doses). In one embodiment, the dose is for use in a human. Kits can include a measured dose for administration to a subject.

An immunogenic composition can be lyophilized. When an immunogenic composition requires reconstitution, it can be provided in the form of a kit which can comprise two vials, or can comprise one ready-filled syringe and one vial, with the contents of the syringe being used to reconstitute the contents of the vial prior to injection.

The disclosed compositions be used in conjunction with other agents, such as another vaccine or therapeutic agent. In some embodiments, the vaccine can be a meningococcal vaccine, such as a conjugate vaccine or a polysaccharide vaccine. In specific non-limiting examples, the vaccine is MPSV4 (MENOMUNE®), MCV4 (MENACTRA®, MENHIBRIX®, MENVEO®) or a serogroup B meningococcal vaccine (TRUMENBA® and BEXSERO®). Additional vaccines are MENCEVAX®, a purified polysaccharide vaccine, such as NmVac4-A/C/Y/W-135, and NIMENTRIX®.

In some embodiments, a single dose is used. In other embodiments, multiple doses are used, such as in a prime boost protocol. Exemplary non-limiting protocols are shown in the examples section. An initial dose and an additional dose can be administered within days, weeks, or months of each other. The initial administration of the mixture can be followed by booster immunization of the same of different mixture, with at least one booster, such as two boosters. The method can include administering 1, 2, 3, 4, 5, 6, 7, 8, 9 or 10 doses.

A subject can be selected for treatment. In some embodiments, the subject has a Neisseriagonorrhoeae infection, and administration of the immunogenic composition increases clearance of the Neisseriagonorrhoeae. In further embodiments, the subject is a healthy subject, and does not have a Neisseriameningitidis or a Neisseriagonorrhoeae infection. In some embodiments, methods are provided for inducing an immune response to Neisseriagonorrhoeae in a mammalian subject, comprising administering to the mammalian subject an immunogenic composition as disclosed herein. The Neisseriagonorrhoeae can be of any serotype. In some embodiments, the subject is a female, and the method induces an immune response at the vaginal mucosa.

The methods include administration of a MetQ protein and a K-type CpG ODN, to a mammalian subject (e.g., a human) to elicit an immune response. The immune response can be against more than one strain of Neisseria species bacteria, and thus protection against disease caused by such bacteria. The disclosed methods can provide for an immunoprotective immune response against a 1, 2, 3, 4, 5 or more strains.

In some embodiments, an immunogenic composition can be administered orally, nasally, nasopharyngeally, parenterally, enterically, gastrically, topically, transdermally, subcutaneously, intramuscularly, in tablet, solid, powdered, liquid, aerosol form, locally or systemically, with or without added excipients. Actual methods for preparing parenterally administrable compositions are described in such publications as Remington’s Pharmaceutical Science, 15th ed., Mack Publishing Company, Easton, Pa. (1980). For oral administration, the compositions may need to be protected from digestion. This is typically accomplished either by association of the composition with an agent that renders it resistant to acidic and enzymatic hydrolysis or by packaging the composition in an appropriately resistant carrier. Means of protecting from digestion are well known in the art.

The MetQ protein and the CpG ODN can be administered to an animal that has or is at risk for acquiring a Neisseriagonorrhoeae infection, to prevent or at least partially arrest the development of disease and its complications. Administration that elicits an immune response to reduce or prevent a Neisseriagonorrhoeae infection, can, but does not necessarily completely, eliminate such an infection, so long as the infection is measurably diminished. For example, administration of an effective amount of the agent can decrease the infection (for example, as measured by number of bacteria, or by number or percentage of subjects infected by a desired amount, for example by at least 10%, at least 20%, at least 50%, at least 60%, at least 70%, at least 80%, at least 90%, at least 95%, at least 98%, or even at least 100% (elimination or prevention of detectable Neisseriagonorrhoeae infection) as compared to a suitable control.

In some embodiments, administration is initiated prior to the first sign of disease symptoms, or at the first sign of possible or actual exposure to pathogenic Neisseria. Without being bound by theory, immunoprotective antibodies for N.gonorrhoeae can be generated by immunization with an immunogenic composition.

### EXAMPLES

The high global prevalence of gonorrhea infections, serious consequences to reproductive and neonatal health, and resistance to available antibiotics necessitate development of effective vaccines. Reverse vaccinology strategies identified NGO2139 and homologous GNA1946 (NMB1946) as gonococcal and meningococcal vaccine candidates, respectively. MetQ is a surface-exposed L-methionine binding lipoprotein expressed by a diverse array of Neisseria species. MetQ also elicits bactericidal and functional antibodies. MetQ conservation as assessed. In addition, its function in Ng pathogenesis as determined. Furthermore, the work disclosed herein documents, its use as a gonorrhea protein subunit vaccine formulated with CpG, which signals TLR-9 to induce T helper (Th) 1 responses, using a female murine model of lower genital tract infection. The results presented herein revealed that MetQ is exceptionally well conserved and readily expressed by Neisseriagonorrhoeae in vivo but was not beneficial during competitive infection. Mice immunized with rMetQ-CpG (n=40) exhibited a robust, specific antibody response in both serum and vaginal secretions. Enumeration of IgA and IgG subtypes indicated that all immunoglobulins in vaccinated mice were significantly higher than in unimmunized or adjuvant-only animals. Combined data from two independent challenge experiments showed that mice immunized with rMetQ-CpG cleared infections significantly faster than those given vehicle (p<0.0001) or adjuvant alone (p=0.0013). The gonococcal burden was also significantly lower in rMetQ-CpG immunized mice in comparison to mice given either CpG or vehicle (p=0.02 and p<0.0001). Thus, it was documented that rMetQ-CpG induces a protective immune response that accelerates bacterial clearance from the murine lower genital tract and therefore represents an attractive gonorrhea subunit vaccine.

### Example 1

### MetQ Is Highly Conserved Among Neisseria

MetQ polymorphic sites were analyzed between 36 genetically and temporally diverse Neisseriagonorrhoeae (Ng) isolates including the 2016 World Health Organization reference strains. MetQ had only 2 (0.23%) and 0 variations at the DNA and protein levels, respectively, and was the most conserved vaccine antigen compared to LptD, BamA, TamA, and NGO2054 (Zielke et al., Mol Cell Proteomics 15, 2338-2355 (2016)). To further explore the feasibility of including MetQ in a rationally designed gonorrhea vaccine, this antigen’s prevalence and sequence variability was comprehensively examined using all available Neisseria genome sequences deposited to the PubMLST database and their predicted amino acid sequences. Overall, these investigations corroborated that MetQ was highly conserved, with 23 alleles among 4,411 Ng isolates, which accounted for 50 nucleotide polymorphic sites and 17 amino acid polymorphisms. Remarkably, a single amino acid sequence, represented by 12 of the 23 alleles, accounts for nearly 97% of metQ sequence variation across the Ng isolates deposited in the database, see the table below:

TABLE 1

Analysis of MetQ alleles and number of Ng isolates per allele grouping

| Allele | # of Ng Isolates | % of Total Ng Isolates (n = 4,411) | # of Divergent AAs compared to allele 10 |
|---|---|---|---|
| 10 | 2216 | 50.24% | 0 |
| 8 | 1055 | 23.92% | 0 |
| 171 | 582 | 13.19% | 0 |
| 45 | 377 | 8.55% | 0 |
| 266 | 115 | 2.61% | 1 |
| 246 | 25 | 0.57% | 0 |
| 608 | 8 | 0.18% | 0 |
| 250 | 5 | 0.11% | 1 |
| 248 | 4 | 0.09% | 0 |
| 252 | 4 | 0.09% | 1 |
| 172 | 3 | 0.07% | 1 |
| 41 | 2 | 0.05% | 8 (including 1 gap) |
| 191 | 2 | 0.05% | 0 |
| 249 | 2 | 0.05% | 1 |
| 253 | 2 | 0.05% | 1 |
| 475 | 2 | 0.05% | 1 |
| 247 | 1 | 0.02% | 1 |
| 251 | 1 | 0.02% | 1 |
| 294 | 1 | 0.02% | 0 |
| 437 | 1 | 0.02% | 0 |
| 473 | 1 | 0.02% | 0 |
| 474 | 1 | 0.02% | 1 |
| 550 | 1 | 0.02% | 0 |

For this work, all Ng nucleic acid metQ alleles present in the Neisseria PubMLST database as of Feb. 22, 2019, sorted by prevalence. Percentages calculated according to the total number of Ng isolates with metQ sequence information deposited into the database. Translations of the nucleic acid equences were aligned and the amino acid sequences were compared to the most common allele (10) to assess overall amino acid conservation.

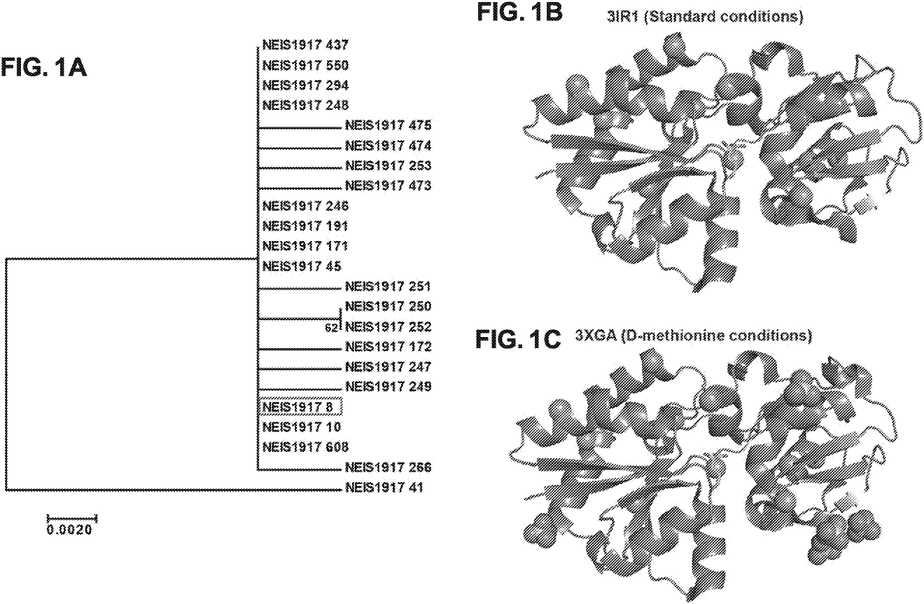

A phylogenetic analysis indicated that all of the MetQ alleles were closely related, with the exception of allele 41, which formed an outgroup (FIG. 1A). Polyclonal antisera elicited by recombinant MetQ (rMetQ) representing allele 8 (Ng FA1090) detected MetQ in whole-cell lysates of diverse Ng isolates, confirming the ubiquitous nature of this antigen and the conservation of epitopes recognized by the immune system ( Zielke et al., Mol Cell Proteomics 15, 2338-2355 (2016)).

A subsequent broader investigation (n=17,613) of other Neisseria isolates that have metQ sequence information in the database revealed 361 metQ alleles, which accounted for 640 and 193 nucleotide and amino acid polymorphic sites, respectively. Similar to Ng, metQ alleles across Neisseria were closely related. Mapping Ng amino acid polymorphisms to N.meningitidis MetQ structures [3XGA and 3IR1; (Yang et al., Journal of structural biology 168, 437-443 (2009))] denoted the presence of 11 low-frequency polymorphisms distributed across the protein (FIGS. 1B, C), none of which is involved in orienting L-methionine in the binding pocket (Yang et al., Journal of structural biology 168, 437-443 (2009)). Furthermore, none of the polymorphic amino acids is present in any of the four most common nucleotide alleles. The reason for the different numbers of polymorphisms is that the first six polymorphic sites are in a region of the protein that was not crystallized. The crystal structure sequence starts at site 44, and the polymorphisms are at sites 2, 27, 33, 35 (gap site), 36, and 42.

These investigations, performed for the first time on a large scale, demonstrate the exceptionally high level of MetQ conservation. Thus, it can serve as a broad-spectrum gonorrhea vaccine and/or next generation vaccines that target both Ng and Nm.

### Example 2

### MetQ is Expressed in Vivo but does not Confer a Detectable Advantage During Competitive Murine Infection

Prokaryotic lipoproteins play versatile functions ranging from cell envelope stability to nutrient acquisition, substrate binding for ABC transporter systems, modulation of the host immune system, signal transduction, and virulence (Kovacs-Simon et al.Infect Immun 79, 548-561 (2011); Hayashi and Wu, PLoS pathogens 14, e1007081 (2018)). In addition to its predicted role in bacterial physiology as a methionine transporter, MetQ may contribute to gonococcal pathogenesis based on the report that a Ng ΔmetQ mutant was attenuated during exposure to primary monocytes and activated macrophages and was less able to adhere to and invade human cervical epithelial cells (Semchenko et al., Infect Immun 85, (2017)). The henotypes associated with deletion of metQ were explored. A null mutant in ngo2139 ΔmetQ) and its complemented mutant ΔmetQ/P_{lac}::metQ in Ng FA1090 were constructed (Zielke et al., Mol Cell Proteomics 15, 2338-2355 (2016)). It was first examined whether complete elimination of MetQ affects cell envelope homeostasis by exposing bacteria to seven antibiotics with different mechanisms of action using Etest assays. These studies showed that Ng lacking MetQ had the same susceptibility as the parental strain (Table 2), suggesting that this lipoprotein has no significant function in cell envelope stability.

TABLE 2

Etest antibiotic susceptibility experiments

| | WT^{a} | ΔmetQ^{a} | ΔmetQ/P_{lac}::metQ^{a} |
|---|---|---|---|
| Polymyxin B | 64 | 64 | 64 |
| Vancomycin | 8 | 8 | 8 |
| Azithromycin | 0.032 | 0.032 | 0.5^{b} |
| Cefotaxime | 0.004 | 0.004 | 0.008 |
| Ampicillin | 0.125 | 0.125 | 0.125 |
| Tetracycline | 0.125 | 0.125 | 0.125 |
| Benzylpenicillin | 0.064 | 0.064 | 0.064 |

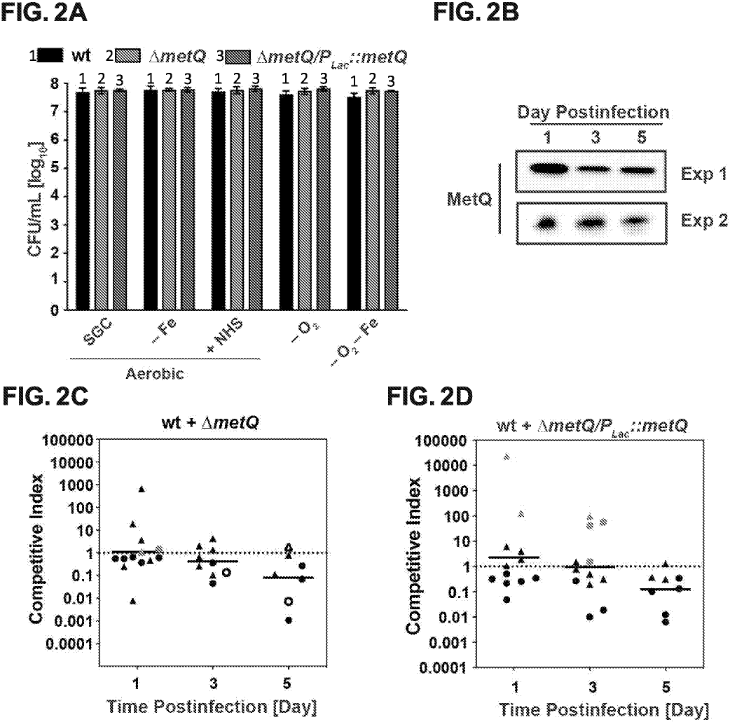

Subsequently, the role of MetQ was investigated during conditions that mimic environmental microniches in the host by exposing WT, ΔmetQ, and the complementation strain ΔmetQ/P_{lac}::metQ to iron limitation, normal human serum, anaerobiosis, and a combination of iron limitation and anoxia. Loss of MetQ did not significantly alter bacterial viability as assessed by colony forming unit (CFU) enumeration compared to the WT strain under any conditions examined (FIG. 2A).

It was demonstrated that MetQ is ubiquitously expressed in a wide range of Ng isolates, throughout different growth phases in liquid medium, and under host-relevant growth conditions (Zielke et al., Mol Cell Proteomics 15, 2338-2355 (2016); Zielke et al., PLoS pathogens 14, e1007081 (2018)). However, its cellular pools present during infection, an important attribute of a promising vaccine candidate antigen, have not been previously been investigated. Female BALB/c mice were infected with WT FA1090 and vaginal washes were collected at days 1, 3, and 5 post-infection in biological duplicate experiments. Samples containing equal numbers of viable Ng bacteria were separated by SDS-PAGE and probed with anti-MetQ antisera. MetQ was readily detectable at each point examined during the infection period (FIG. 2B). Densitometry analyses using MetQ abundance on day 1 as a reference showed that MetQ levels varied slightly on day 3 post-infection (0.91 ± 0.41; mean ± SEM) and lowered to 0.76-fold (±0.14; SEM) on day 5. This studied indicates that MetQ expression in vivo is relatively stable during experimental infection.

Finally, to assess whether MetQ provides Ng with a fitness advantage during experimental murine infection, competitive infection experiments were performed in which mice were inoculated vaginally with similar numbers of WT FA1090 mixed with either the ΔmetQ mutant or the ΔmetQ/P_{lac}::metQ complementation strain. The calculated competitive indices (CIs) for ΔmetQ/WT were 1.09, 0.42, and 0.08 (geometric means of biological duplicate experiments) on day 1, 3, and 5 post-infection, respectively (FIG. 2C). A similar decrease in CI over time was observed for the ΔmetQ/P_{lac}::metQ/WT competition (FIG. 2D). Statistical analysis of the data revealed no significant differences. Calculated p values for competition experiments conducted in vitro were also not significant.

Thus, MetQ is produced in vivo but does not provide a growth advantage in vitro or a growth or survival advantage during mucosal infection in the lower genital tract of female mice.

### Example 3

### MetQ as a Vaccine Component

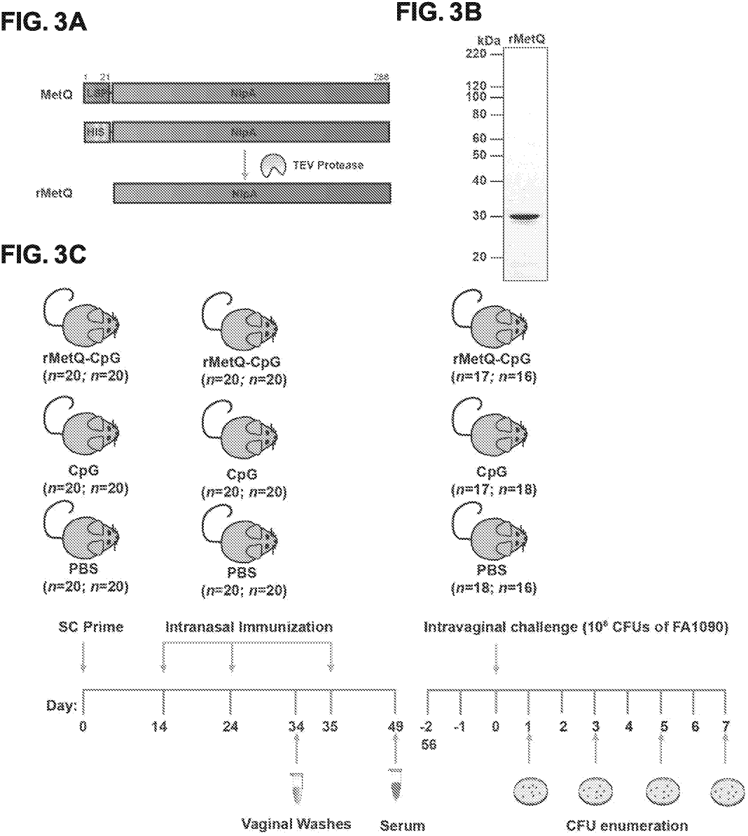

To appraise MetQ as a component of gonorrhea subunit vaccines, a recombinant protein construct was designed using the highly prevalent allele 8 from Ng FA1090 (Table 1) by replacing the MetQ lipoprotein signal peptide with an N-terminal 6xhistidine tag (FIG. 3A). A highly pure untagged rMetQ antigen that migrated on SDS-PAGE with a predicted molecular weight of 29.87 kDa, which corresponds to mature MetQ, was obtained after two-step chromatography coupled to Tobacco Etch Virus (TEV) protease cleavage (FIG. 3B). Note that the protein sequence is shown in FIG. 8.

Vaccine development against Ng is challenging, due partially to the lack of established correlates of protection, as well as the pathogen’s ability to evade the adaptive response through suppression of Th1- and Th2-cell proliferation and the induction of regulatory T cells (Zhu et al., PloS one 7, e41260 (2012); Zhu et al., J Biol Chem 293, 11218-11229 (2018); Liu et al., Mucosal immunology 7, 165-176 (2014); Feinen et al., Mucosal immunology 3, 312-321 (2010)). Ng-mediated suppression of Th1- and Th2- cells during experimental murine infection, which occurs through a TGF-β-dependent mechanism, can be reversed by administration of anti-TGF-β, anti-IL-10, or IL-12, resulting in a humoral memory response and protection from rechallenge (Liu et al., Mucosal immunology 7, 165-176 (2014); Liu and Russell, mBio 2, e00095-00011 (2011); Liu et al., mSphere 3, (2018); Liu et al., Mucosal immunology 10, 1594-1608 (2017)). It was postulated that Th1 responses is critical for protection against Ng. Therefore, to assess the protective potential of rMetQ in vivo, a pilot experiment was conducted, followed by two immunization/challenge studies using rMetQ formulated with CpG (FIG. 3C), which is the FDA-approved adjuvant used in the Heplisav-B subunit vaccine against Hepatitis B.

First, the immunogenicity of rMetQ was tested by immunizing groups of 5 BALB/c mice with rMetQ, rMetQ-CpG, CpG, or PBS using a subcutaneous prime immunization, followed by a regimen of three intranasal boost doses. A robust, single protein band corresponding to the molecular weight of rMetQ (~30 kDa) was detected in immunoblots with serum derived from mice immunized with rMetQ alone. Immune recognition was slightly enhanced when antisera to rMetQ-CpG were used (44,405 versus 49,893 relative intensity units, respectively), indicating that rMetQ is a strong immunogen on its own. No signal was detected in sera obtained from animals immunized with adjuvant alone or PBS. Based on these encouraging results, large-scale immunization/challenge experiments were performed. To ensure sufficient statistical power for pre-clinical vaccine research, cohorts (n=20/group) of BALB/c mice were given rMetQ formulated with CpG, adjuvant alone, or PBS in biological duplicate experiments (FIG. 3C). Vaginal washes were collected after the second boost, and antigen-specific antibody titers were assessed in serum collected 14 days after the third boost. Three weeks after the final immunization, solely those mice that entered the diestrus or anestrus phase (34) were challenged with Ng FA1090 using a well-established infection protocol (35). Bacterial clearance was assessed by enumerating Ng CFUs present in vaginal washes on days 1, 3, 5, and 7 post-infection (FIG. 3C).

### Example 4

### MetQ-CpG Elicits aProminent Antigen-Specific Th1 Response

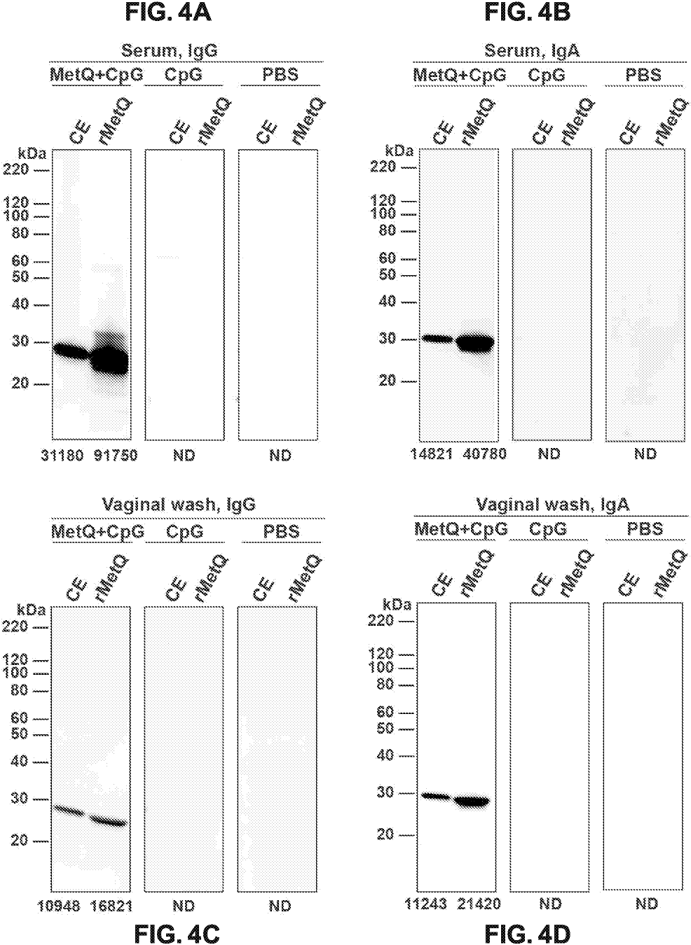

The immunization regimen induced a strong MetQ-specific serum and vaginal antibody response as assessed by immunoblot of Ng cell envelopes (CE) and rMetQ using serum and vaginal washes from immunized and control mice, detected with secondary anti-mouse IgG or IgA (FIGS. 4A-C). Serum and vaginal IgG (FIGS. 4A and B, respectively) as well as vaginal IgA (FIG. 4C) obtained from mice immunized with rMetQ-CpG readily recognized a single ~30 kDa protein band in the CE samples that aligned with the rMetQ band. Overall, relative MetQ intensities were lower in samples probed with vaginal secretions than with serum. No signal was detected when CE or rMetQ were probed with serum or vaginal wash immunoglobulins obtained from control mice (CpG- or PBS-immunized). Additionally, relative intensities of 1 µg of purified rMetQ in comparison to MetQ present in a crude CE protein fraction when probed with serum IgG, vaginal IgG and IgA secretions were only 1.98-, 1.58- and 2.26-fold higher, suggesting that robust MetQ pools reside in the Ng cell envelope. Together, these results demonstrated that immunization of mice with rMetQ-CpG induces antigen-specific immune responses that are both systemic and, critically, at the genital mucosae.

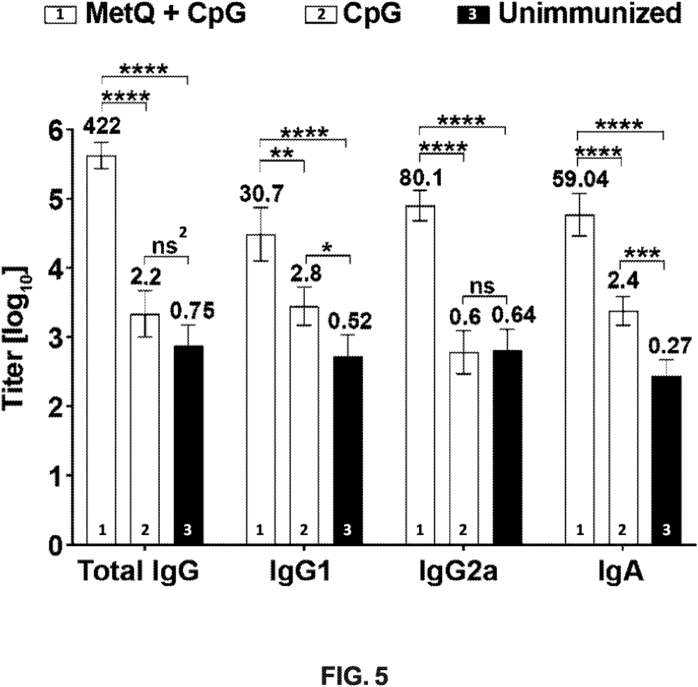

Titers of rMetQ-specific antibodies were measured by ELISA. Analyses of the non-transformed combined ELISA data from both immunization experiments showed that total serum IgG and IgA titers in mice immunized with rMetQ-CpG were significantly higher than in mice that received adjuvant alone or PBS, as were IgG1 and IgG2a titers (FIG. 5). The calculated geometric mean of total IgG in mice immunized with rMetQ-CpG was 4.2×10⁵ in comparison to 2.2×10³ and 7.5×10² in mice that received CpG and PBS, respectively. For IgGl the values were 3.1×10⁴, 2.8×10³, and 5.2×10² for mice immunized with rMetQ-CpG, CpG alone, and PBS, respectively. IgG2a antibody levels were significantly (133-fold) higher in mice that received rMetQ-CpG (8.0×10⁴) versus both control groups (6.1 × 10² for both groups). Finally, the titers for IgA were 24.6- and 218-fold higher in rMetQ-CpG-immunized mice than in CpG and PBS control groups, respectively. The IgGl/IgG2a ratio of 0.38 indicates a slight bias toward a Th1 response, consistent with the activity of CpG as a Thl-stimulating adjuvant.

Cumulatively, these analyses demonstrated that rMetQ-CpG formulation elicits significantly high antibody titers and high levels of IgG2a.

### Example 5

### Immunization With rMetQ-CpG Accelerates Clearance From Experimentally Challenged Mice

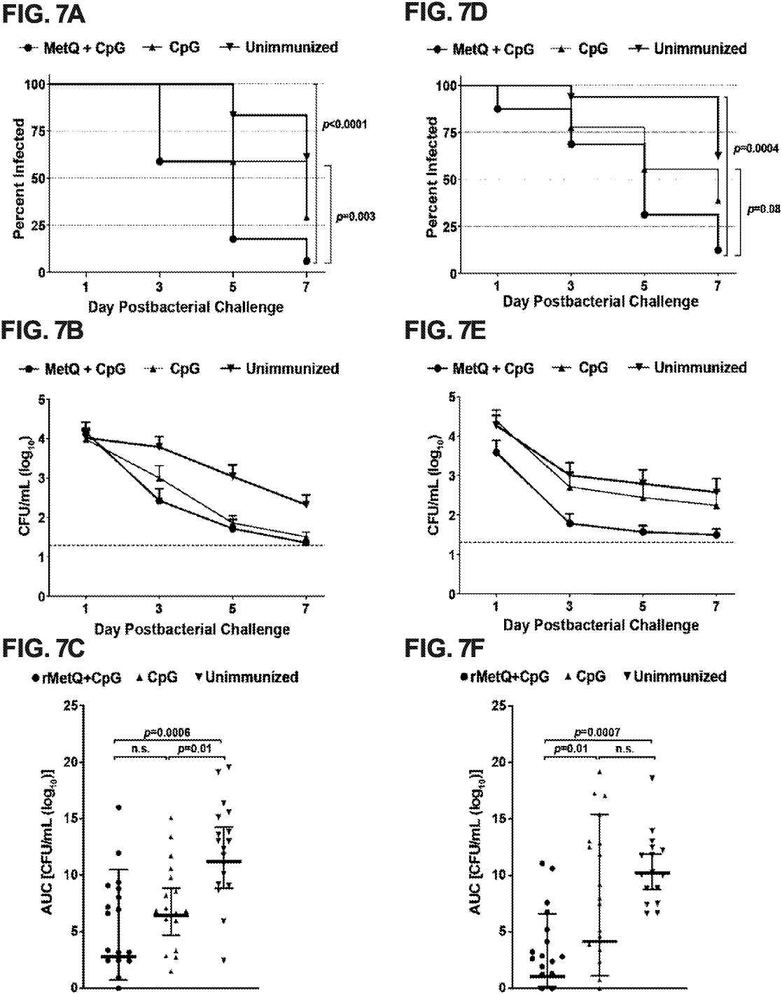

To test whether rMetQ-CpG could induce a protective response, immunized and control mice were challenged vaginally with WT FA1090 Ng. Vaginal swabs were quantitatively cultured for Ng on days 1, 3, 5, and 7 after challenge (FIG. 3C). Infection duration and bacterial burden were compared between immunized and control groups on two separate occasions (FIGS. 7A-7F). In the first experiment, comparison of the percentage of culture-positive mice over time in each group showed that rMetQ-CpG-immunized mice had a significantly faster clearance rate compared to mice given PBS (p < 0.0001) or adjuvant alone (p ≤ 0.003; FIG. 7A). The recovered bioburden was similar between immunized mice and mice given adjuvant alone (FIGS. 7B-C). A comparison of area under curve (AUC), a measure of the cumulative burden of infection over time, showed that both immunized and adjuvant-alone groups were significantly different than unimmunized mice (PBS) (p = 0.0006 and 0.01, respectively; FIG. 7C). In the repeat experiment, faster clearance was also observed in the MetQ-immunized mice compared to the unimmunized group (p = 0.0004; FIG. 7D). Mice immunized with rMetQ-CpG had a 4- and 10-fold lower AUC than mice given either adjuvant alone or PBS (p = 0.01 and p = 0.0007, respectively; FIG. 7F). No protective effect of the adjuvant alone was detected in this experiment.

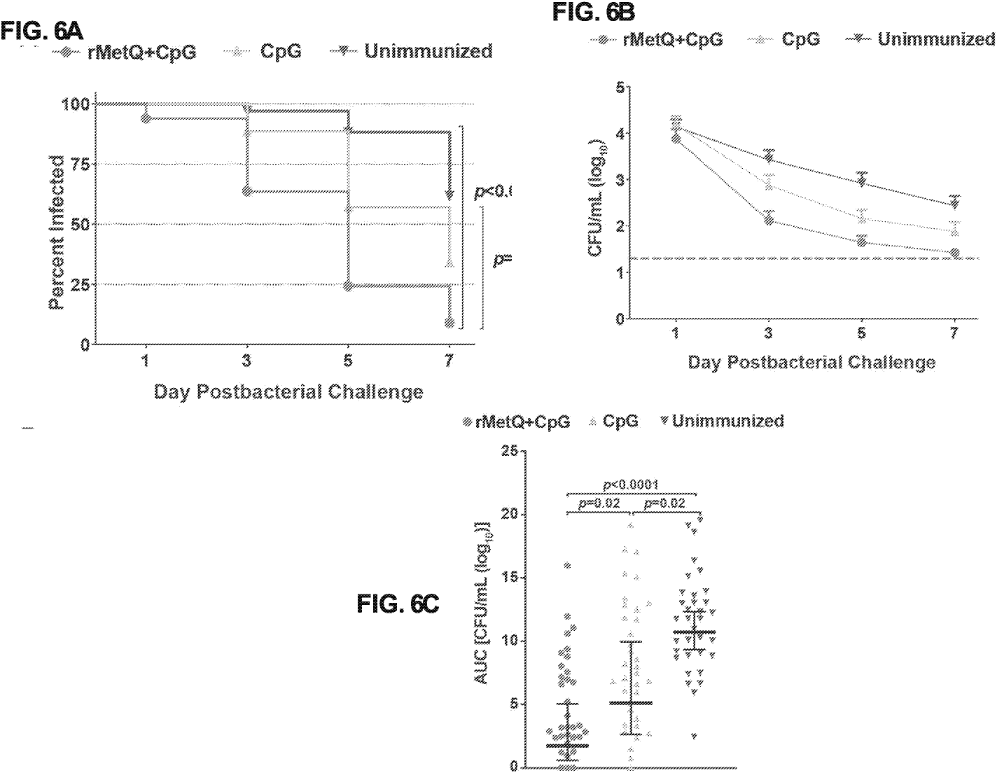

The combined data for the two MetQ challenge experiments are shown in FIG. 6. Mice immunized with rMetQ-CpG cleared the infections significantly faster than those given PBS (p<0.0001) or adjuvant alone (p=0.0013; FIG. 6A). The gonococcal burden was also significantly lower in rMetQ-CpG immunized mice in comparison to mice given either CpG or PBS (p=0.02 and p<0.0001, respectively; FIGS. 6B, C). Importantly, by day 5 and 7 post-challenge, 75.5 and 90.8% of mice that received rMetQ-CpG cleared gonorrhea infection, compared to 42.8 and 65.8% for CpG-inoculated animals and 11.4 and 38.19% in the placebo group, respectively.

In summary, MetQ formulated with CpG induced a protective immune response that accelerated Ng clearance from the murine lower genital tract.

In recent years, an increasing number of reports have evaluated the ability of gonorrhea vaccine candidates to elicit immune responses in animals. However, very few reports demonstrated the induction of a protective response that would enhance clearance and shorten the duration of infection (Rice et al., Annual review of microbiology 71, 665-686 (2017).). In the present study, reverse vaccinology: MetQ [NG02139; (Zielke et al., Mol Cell Proteomics 15, 2338-2355 (2016); Zielke et al., Mol Cell Proteomics 13, 1299-1317 (2014); Pizza et al., Science 287, 1816-1820 (2000); Semchenko et al., Infect Immun 85, (2017))] was used, and a lipoprotein antigen identified that induced an immune response and had protective capability. In light of the extensive surface protein variability inherent to Ng, highly conserved antigens must be selected to provide as broad coverage as possible. Using extensive bioinformatics analyses it was determined that MetQ was a desirable antigen based on its conservation: a single amino acid sequence accounts for nearly 97% of global Ng MetQ variation (FIG. 1, Table 1). Thus, vaccination with a single MetQ variant can provide broad protection against the majority of Ng encountered worldwide.

Conservation data from Nm suggest that MetQ may be a suitable antigen for a universal Neisseria vaccine. Mice immunized with a highly pure recombinant MetQ variant, rMetQ, formulated with CpG exhibited a robust, specific antibody response in both serum and vaginal secretions (FIG. 4). The presence of MetQ-specific IgA and IgG in the vaginal mucosae suggests that vaccination results in an antibody response at the site of infection. Enumeration of IgA and IgG subtypes indicated that all immunoglobulins in vaccinated mice were significantly higher than in unimmunized or adjuvant-only animals (FIG. 5).

The induction of a Th1 response is likely to be important for effective gonococcal clearance, as demonstrated by the protective capabilities of experimental vaccines formulated with Thl-inducing adjuvants and delivery systems (Liu et al., mSphere 3, (2018, 30-33); Zhu et al., Frontiers in microbiology 2, 124 (2011); Gulati et al., PLoS pathogens 9, e1003559 (2013); Amanda DeRocco et al., International Pathogenic Neisseria Conference, (2014); Zhu et al., Infect Immun 73, 7558-7568 (2005)). The Th1 bias resulting from immunization with the disclosed rMetQ was indicated by an IgGl/IgG2a ratio of 0.38. rMetQ-CpG vaccination both significantly accelerated clearance (FIG. 6A) and reduced bacterial burden in infected animals (FIG. 6B). Together, these data provide compelling evidence that MetQ will be a valuable component of a protective subunit vaccine against gonorrhea.

Deletion of MetQ is minimally detrimental either in vitro under conditions relevant to infection (FIG. 2A) or during in vivo competitive infections (FIGS. 2C and D). Thus, the bactericidal activity of anti-rMetQ antibodies is more likely to contribute to the vaccine’s protective ability.

It was demonstrated that immunization with rMetQ formulated with CpG induces a robust, specific Th1 response against rMetQ, accompanied by generation of vaginal mucosae and serum antibodies, and significantly shortens gonococcal infection. The looming threat of untreatable gonorrhea requires new weapons to fight its spread. A vaccine built around the highly conserved, protective antigen MetQ would transform human reproductive health by eradicating an ancient, worldwide public health scourge.

### Example 6

### Additional Comparative Studies

FIG. 11 presents data from individual immunization/challenge studies. A cohort of mice received rMetQ alone. The percentage of culture-positive mice over time showed that rMetQ-CpG-immunized mice had a significantly faster clearance rate compared to mice given rMetQ (p=0.02) or PBS (p=0.001) but not adjuvant alone (FIG. 11A). The cumulative burden of infection over time was significantly lower in rMetQ-CpG-immunized mice compared to the rMetQ-, CpG- and PBS-groups (p=0.005, p=0.03 and p=0.003, respectively; FIG. 11C). No significant differences were observed in CpG-treated mice compared to mice that received PBS (FIGS. 11A-C ). A cohort of mice given rMetQ showed no difference in the clearance rate or bioburden compared to control groups (FIGS. 11A-11C). Thus, formulation with CpG is required for MetQ to confer protection.

An additional gonorrhea vaccine candidate, NGO1985, was formulated as a recombinant protein with CpG as an adjuvant. The same immunization schedule and experimental outline was followed as for MetQ-CpG. A single large-scale immunization/challenge experiment was performed with cohorts (n=20/group) of BALB/c mice that were given NGO1985 formulated with CpG, adjuvant alone, or PBS and challenged vaginally with wild type FA1090 Ng three weeks after the final immunization. Bacterial clearance was assessed by enumerating Ng CFUs present in vaginal washes on days 1, 3, 5, and 7 post-infection (FIG. 12A). Infection duration and bacterial burden were compared between immunized and control groups (FIG. 12B). There were no significant differences in infection duration and bacterial burden between mice immunized with NGO1985-CpG and control groups that received either CpG or PBS (FIGS. 12A-12B). This experiment clearly showed that another antigen, NGO1985 formulated with CpG, was not protective against Neisseriagonorrhoeae infection.

### Example 7

### Materials and Methods

MetQ bioinformatics analyses: Polymorphism, phylogenetic, and allele mapping analyses were performed as described by (Baarda et al., Frontiers in microbiology 9, 2971 (2018)). Briefly, the nucleotide sequence of metQ (ngo2139) was used to query the public Neisseria multilocus sequence typing database ( Neisseria pubMLST; (Jolley et al., Wellcome Open Res 3, 124 (2018)); pubmlst.org/neisseria/) to identify metQ alleles and nucleotide polymorphic sites across 4,411 isolates of N.gonorrhoeae for which metQ sequence data exist, as well as among all Neisseria isolates present in the database (17,613 isolates with metQ sequence data as of Feb. 22, 2019). Translated amino acid sequences were aligned with ClustalW in MEGA7 (Kumar et al., Mol Biol Evol 33, 1870-1874 (2016)). Alignments were used to generate a maximum likelihood phylogenetic tree. The Jones-Taylor-Thornton model (Jones et al., Comput Appl Biosci 8, 275-282 (1992) was used to generate a pairwise distance matrix, to which Neighbor-Join and BioNJ algorithms were applied to generate the initial tree. The Nearest-Neighbor-Interchange method was employed to heuristically search the initial tree. Five hundred bootstrap replications were performed to test the phylogenies. Amino acid polymorphisms were mapped to crystal structures of the N.meningitidis MetQ homolog, GNA1946. The amino acid sequences of structures solved from proteins isolated after propagation in rich medium (3GXA; (Yang et al., Journal of structural biology 168, 437-443 (2009))) and minimal medium with D-methionine as the sole methionine input (3IR1; (Yang et al., Journal of structural biology 168, 437-443 (2009))) were aligned against all Ng MetQ amino acid sequences as above. Polymorphic sites were identified and their prevalence was calculated by dividing the number of variants with polymorphisms by the total number of variants. The polymorphic sites were then mapped to the crystal structure using PyMOL (pymol.org/2/) and colored according to their prevalence. The prevalence was not weighted by the number of isolates with each particular polymorphism.

Etest antimicrobial sensitivity assessments: The susceptibility of WT FA1090, isogenic knockout ΔmetQ, and complementation strain ΔmetQ/P_{lac}: :metQ to seven antimicrobial compounds was assessed by Etests as previously (Zielke et al., PLoS pathogens 14, e1007081 (2018)), according to manufacturer’s instructions. Briefly, non-piliated colonies of each strain were suspended in GCBL to a turbidity equivalent to that of a 0.5 McFarland standard and spread on 150 mm tissue culture dishes filled with 50 mL GCB to a thickness of 4 mm. Etest strips were placed on the agar surface and plates were cultured for ~22 h, at which point the MICs were determined. Sensitivity assessments were performed three times, and MIC values that repeated at least twice are reported.

Isolation of Ng crude cell envelope fractions: Total cell envelope (CE) fractions were prepared from mid-logarithmic cultures of WT Ng FA10190 by sonication and ultracentrifugation. Briefly, bacteria were cultured in supplemented GC broth to an A₆₀₀ of 0.6, collected, resuspended in PBS and disrupted by sonication. Cell debris and remaining intact cells were removed by centrifugation. Total cell envelope fraction was obtained from crude cell lysates by ultracentrifugation, resuspended in PBS and frozen at -20° C. Protein concentration was measured using a NanoDrop Spectrophotometer (ND-1000).

Immunoblotting and SyproRuby staining: Samples of purified MetQ (1 µg), crude cell envelope fractions (CE; 1 µg), or vaginal washes (normalized based on CFUs) were fractionated by SDS-PAGE, transferred onto nitrocellulose membranes and detected by immunoblotting as described previously (Zielke et al., Mol Cell Proteomics, (2016). Protein concentration was measured using the DC Protein Assay (BioRad). The immunoblotting analysis of MetQ expression during murine infection was performed using polyclonal rabbit antisera against MetQ (Zielke et al., Mol Cell Proteomics, (2016), and secondary anti-goat anti-rabbit HRP conjugated antibodies (Bio-Rad). For experiments assessing specificity of murine sera and vaginal washes, membranes were blocked overnight in PBST supplemented with 5% non-fat dry milk and incubated with antiserum (1:5,000) or vaginal washes (1:500) from test or control mice followed by goat anti-mouse IgG (BioRad) or IgA (SouthernBiotech) conjugated to HRP. Washes between incubations were performed with PBST. Membranes were developed using ECL Prime (Amersham) and IMAGEQUANT™ LAS 4000 (GE Healthcare). Proteins were visualized with SyproRuby (BioRad) per manufacturer’s recommendations.

Densitometry analysis: The expression of MetQ during WT FA1090 infection in female BALB/c mice and intensities of protein bands detected in serum specificity studies were determined by densitometry using FIJI software (Schindelin et al., Nature methods 9, 676-682 (2012)). The amount of MetQ on day 1 post-infection was arbitrarily set to 1 and the protein abundance on days 3 and 5 is expressed relative to the MetQ cellular pool detected on day 1.

Statistical analysis: GraphPad Prism was used for all statistical analyses. The built-in t-test was utilized to determine statistically significant differences between experimental results with the exception of animal studies and ELISA, which were analyzed as described above. A confidence level of 95% was used for all analyses.

In view of the many possible embodiments to which the principles of our invention may be applied, it should be recognized that illustrated embodiments are only examples of the invention and should not be considered a limitation on the scope of the invention. Rather, the scope of the invention is defined by the following claims. We therefore claim as our invention all that comes within the scope and spirit of these claims.

1. A method of inducing an immune response to Neisseria gonorrhoeae in a mammalian subject, comprising
administering to the mammalian subject an effective amount of a MetQ protein and an effective amount of a K-type CpG oligodeoxynucleotide,
thereby inducing the immune response.

2. The method of claim 1, wherein the K-type CpG ODN comprises the consensus nucleotide sequence of SEQ ID NO: 7, such as SEQ ID NO: 38, or a nucleotide sequence set forth as any one of SEQ ID NOs: 8-42.

3. The method of claim 1, wherein the K-type CpG ODN comprises the nucleotide sequence of SEQ ID NO: 38, SEQ ID NO: 39, SEQ ID NO: 40, SEQ ID NO: 41 or SEQ ID NO: 42.

4. The method of claim 1, wherein the MetQ protein comprises positions 24-289 of SEQ ID NO: 1 with at most 15 amino acid substitutions.

5. The method of claim 4, wherein the MetQ protein comprises one or more of S36A, A42V, A65P, A65V, A119V, A158V, N163D, S220G, D263N, A259V, R272C, Y278S, and N150D.

6. The method of claim 4, wherein the MetQ protein comprises an N-terminal fusion to an amino acid sequence set forth as GAME (SEQ ID NO: 2).

7. The method of claim 1, wherein the MetQ protein comprises an C-terminal fusion to an amino acid sequence set forth as KLAAA (SEQ ID NO: 3).

8. The method of claim 1, wherein the MetQ protein comprises SEQ ID NO: 4.

9. The method of claim 1, wherein the immune response is a protective immune response.

10. The method of claim 1, wherein the immune response is a therapeutic response.

11. The method of claim 10, wherein the subject has a Neisseriagonorrhoeae infection, and administration of the immunogenic composition increases clearance of Neisseriagonorrhoeae.

12. The method of claim 9, wherein the mammalian subject is a healthy subject.

13. The method of claim 1, wherein the mammalian subject is a human.

14. An immunogenic composition comprising an effective amount of a MetQ protein and an effective amount of a K-type CpG oligodeoxynucleotide.

15. The immunogenic composition of claim 14, comprises the consensus nucleotide sequence of SEQ ID NO: 7, such as SEQ ID NO: 38, or a nucleotide sequence set forth as any one of SEQ ID NOs: 8-42.

16. The immunogenic composition of claim 14, wherein the K-type CpG ODN comprises the nucleotide sequence of SEQ ID NO: 38, SEQ ID NO: 42, SEQ ID NO: 39, or SEQ ID NO: 40, SEQ ID NO: 41.

17. The immunogenic composition of claim 14, wherein the MetQ protein is a recombinant MetQ protein.

18. The immunogenic composition of claim 14, wherein the MetQ protein comprises positions 24-289 of SEQ ID NO: 1 with at most 15 amino acid substitutions.

19. The immunogenic composition of claim 18, wherein the MetQ protein comprises one or more of S36A, A42V, A65P, A65V, A119V, A158V, N163D, S220G, D263N, A259V, R272C, Y278S, and N150D.

20. The immunogenic composition of claim 17, wherein the MetQ protein comprises a) an N-terminal fusion to an amino acid sequence and/orb) a C-terminal fusion to anamino sequence set forth as GAME (SEQ ID NO: 2); and/or b) a C-terminal fusion to an amino acid sequence set forth as KLAAA (SEQ ID NO: 3).

21. (canceled)

22. The immunogenic composition of claim 17, wherein the MetQ protein comprises SEQ ID NO: 4.

23-24. (canceled)
